(19) United States
(12) Patent Application Publication
(10) Pub. No.: US 2025/0366942 A1
(43) Pub. Date: Dec. 4, 2025

(54) TABLE-MOUNTED MANIPULATOR SYSTEM, AND RELATED DEVICES, SYSTEMS AND METHODS

(71) Applicant: INTUITIVE SURGICAL OPERATIONS, INC., Sunnyvale, CA (US)

(72) Inventors: Steven Manuel, Sunnyvale, CA (US); Andrew Cullen Waterbury, Sunnyvale, CA (US); Kevin Lau, Fremont, CA (US); Bram Gilbert Antoon Lambrecht, Redwood City, CA (US); Daniel H. Gomez, Los Gatos, CA (US); John Ryan Steger, Los Gatos, CA (US); Allison Grey, San Francisco, CA (US); Amy Mueller, Mountain View, CA (US); Arpit A. Mittal, Redwood City, CA (US)

(73) Assignee: INTUITIVE SURGICAL OPERATIONS, INC., Sunnyvale, CA (US)

(21) Appl. No.: 18/860,958

(22) PCT Filed: Apr. 28, 2023
(86) PCT No.: PCT/US2023/020441

§ 371 Date: Oct. 28, 2024

Related U.S. Application Data
(60) Provisional application No. 63/336,840, filed on Apr. 29, 2022.

(51) Int. Cl. A61B 34/35; A61B 34/30; A61G 13/10; B25J 5/02; B25J 9/04; B25J 15/00; B25J 18/02

(52) U.S. Cl. CPC A61B 34/35; A61G 13/101; B25J 5/02; B25J 9/04; B25J 15/0019; B25J 18/02; A61B 2034/305

(57) ABSTRACT

A teleoperable manipulator system can comprise a table assembly, a rail coupled to the table assembly, and a manipulator coupled to the rail. The table assembly comprises a platform configured to support a body, the platform having lateral and longitudinal dimensions. The manipulator is translatable relative to the rail along a longitudinal dimension of the rail. The manipulator comprises a proximal link assembly comprising a proximal arm coupled to the rail by one or more proximal joints. The proximal arm is extendable in length.

### CROSS-REFERENCE TO RELATED APPLICATIONS

This application claims priority to U.S. Provisional Application No. 63/336,840, entitled “TABLE-MOUNTED MANIPULATOR SYSTEM, AND RELATED DEVICES, SYSTEMS, AND METHODS,” filed Apr. 29, 2022, the entire contents of which is incorporated herein by reference.

### FIELD

Aspects of this disclosure relate generally to table-mounted manipulator systems. In particular, aspects of the disclosure relate to manipulators for table-mounted manipulator systems, such as a medical system table for supporting a patient. Related devices, systems, and methods also are disclosed.

### INTRODUCTION

Computer-assisted manipulator systems (“manipulator systems”), sometimes referred to as robotically assisted systems or robotic systems, can include one or more manipulators that can be operated with the assistance of an electronic controller (e.g., computer) to move and control functions of one or more instruments coupled to the manipulators. A manipulator generally includes mechanical links connected by joints. An instrument is removably couplable to (or permanently coupled to) one of the links, typically a distal link of the plural links.

In some computer-assisted manipulator systems, the manipulators are attached to a manipulator support structure (e.g., a patient side cart) that is separate from a support structure that supports a patient or workpiece. In other manipulator systems, the manipulators are attached directly to the support structure (herein referred to as a “table assembly”) that supports the patient or workpiece, e.g., to an operating table. Manipulator systems in which the manipulators are mounted to the table assembly can be referred to herein as table-mounted manipulator systems.

Table-mounted manipulator systems pose certain challenges. The space around a table assembly can need to be occupied with various pieces of equipment and/or personnel during the performance of various tasks that make up a medical procedure. Moreover, the space constraints around the table can vary depending on the medical procedure being performed, with some tasks (such as transferring a patient to the table, draping manipulators, etc.) benefiting from or being facilitated by a large amount of open space around the table. In manipulator systems including movable patient-side carts, such open space around the table can be obtained by moving the patient-side cart away from the table intended to support the patient. However, in table-mounted manipulator systems, moving the manipulators out of the way when space is desired around the table poses challenges as the manipulators are either affixed to the table or at least not practical to remove from the table during a particular stage of a medical procedure. Thus, it can be challenging to avoid interference between the manipulators and the other entities in the space around the table in a table-mounted manipulator system.

In addition, some table assemblies can switch between multiple spatial configurations, such as by lowering or raising sections of a multi-section table, and it can be challenging to accommodate such changes in configuration of the table assembly when the manipulators are mounted to the table assembly.

Another challenge with table-mounted manipulators is that the positioning of the manipulators relative to a patient can be limited due to the manipulators being attached to the table, thus making it more difficult to reach certain portions of a patient or certain desired angles during a medical procedure. In particular, due to the limited reach of the manipulators coupled to the table, some systems can be relatively limited as to the locations of entry ports that the system can accommodate (entry ports being the natural orifices or incisions through which an instrument enters the patient, for example via a cannula disposed in the entry port).

Accordingly, a need exists for improved table-mounted manipulator systems, in particular systems with improved manipulator architectures.

### SUMMARY

Various embodiments of the present disclosure can solve one or more of the above-mentioned problems and/or can demonstrate one or more of the above-mentioned desirable features. Other features and/or advantages can become apparent from the description that follows.

In accordance with at least one embodiment of the present disclosure a teleoperable manipulator system can include a table assembly, a rail coupled to the table assembly, and a manipulator coupled to the rail. The table assembly can include a platform configured to support a body, the platform having lateral and longitudinal dimensions. The manipulator can be translatable relative to the rail along a longitudinal dimension of the rail. The manipulator can include a proximal link assembly including a proximal arm coupled to the rail by one or more proximal joints. The proximal arm can be extendable in length.

In accordance with at least one embodiment of the present disclosure a teleoperable manipulator system can include a table assembly, a rail coupled to the table assembly, and a manipulator coupled to the rail. The table assembly can include a platform configured to support a body, the platform having lateral and longitudinal dimensions. The manipulator can be translatable relative to the rail along a longitudinal dimension of the rail. The manipulator can include a proximal link assembly including a proximal arm coupled to the rail by one or more proximal joints, and an intermediate link assembly including an intermediate arm coupled to the proximal arm. The intermediate arm can be extendable in length.

In accordance with at least one embodiment of the present disclosure a teleoperable manipulator system can include a table assembly, a rail coupled to the table assembly, and a manipulator coupled to the rail. The table assembly can include a platform configured to support a body, the platform having lateral and longitudinal dimensions. The manipulator can be translatable relative to the rail along a longitudinal dimension of the rail. The manipulator can include a proximal link assembly including a proximal arm coupled to the rail by one or more proximal joints. The one or more proximal joints are configured to provide rotation of the proximal arm about a first axis perpendicular to the proximal arm and about a second axis perpendicular to the first axis and parallel to the rail.

In accordance with at least one embodiment of the present disclosure a teleoperable manipulator system can include a table assembly, a rail coupled to the table assembly, and a manipulator coupled to the rail. The table assembly can include a platform configured to support a body, the platform having lateral and longitudinal dimensions. The manipulator can be translatable relative to the rail along a longitudinal dimension of the rail. The manipulator can be configured to extend above and across the platform to a position adjacent a side of the platform opposite from a side on which the rail can be disposed.

In accordance with at least one embodiment of the present disclosure a teleoperable manipulator system can include a table assembly, a rail coupled to the table assembly, and a manipulator coupled to the rail. The table assembly can include a platform configured to support a body, the platform having lateral and longitudinal dimensions. The manipulator can be translatable relative to the rail along a longitudinal dimension of the rail. The manipulator can include a proximal link assembly, an intermediate link assembly, a distal link assembly, a wrist, and an instrument holding portion. The proximal link assembly can include a proximal arm coupled to the rail by one or more proximal joints. The intermediate link assembly can include an intermediate arm coupled to the proximal arm. The distal link assembly can include a distal arm coupled to the intermediate arm. The wrist can include three rotational degrees of freedom of motion. The instrument holding portion can be configured to mount an instrument thereto and can be coupled to the distal arm by the wrist.

In accordance with at least one embodiment of the present disclosure a teleoperable manipulator system can include a table assembly, a rail coupled to the table assembly, and a manipulator coupled to the rail. The table assembly can include a platform configured to support a body, the platform having lateral and longitudinal dimensions. The manipulator can be translatable relative to the rail along a longitudinal dimension of the rail. The manipulator can include a proximal link assembly, an intermediate link assembly, a distal link assembly, a wrist, and an instrument holding portion. The proximal link assembly can include a proximal arm coupled to the rail by a first proximal joint and a second proximal joint. The first proximal joint can be configured to provide rotation of the proximal arm about a first axis perpendicular to the proximal arm. The second proximal joint configured to provide rotation of the proximal arm about a second axis perpendicular to the first axis and parallel to the rail. The intermediate link assembly can include an intermediate arm coupled to the proximal arm. The distal link assembly can include a distal arm coupled to the intermediate arm. The wrist can include three rotational degrees of freedom of motion. The instrument holding portion can be configured to mount an instrument thereto and can be coupled to the distal arm by the wrist. The proximal arm and the intermediate arm are extendable in length.

In accordance with at least one embodiment of the present disclosure, a medical system can include a table assembly, a rail coupled to the table assembly, and a manipulator movably coupled to the rail. A method of operating this medical system can include positioning the manipulator such that the manipulator extends above a platform of the table assembly and extends across the table assembly from a first longitudinally extending side of the platform to a second longitudinally extending side of the platform opposite from the first longitudinally extending side.

In accordance with at least one embodiment of the present disclosure, a teleoperable manipulator system can include a table assembly, a rail coupled to the table assembly, and a manipulator coupled to the rail. The table assembly can include a platform configured to support a body, the platform having lateral and longitudinal dimensions. The manipulator can be translatable relative to the rail along a longitudinal dimension of the rail. The manipulator can include an arm, a wrist coupled to the arm, an instrument holding portion configured to mount an instrument thereto, and the instrument holding portion being coupled to the arm by the wrist. The wrist can include three rotational degrees of freedom of motion. At least one of the degrees of freedom of motion of the wrist can be a redundant degree of freedom of motion.

In accordance with at least one embodiment of the present disclosure, a teleoperable manipulator system can include a table assembly, a rail coupled to the table assembly, and a manipulator coupled to the rail. The table assembly can include a platform configured to support a body, the platform having lateral and longitudinal dimensions. The manipulator can be translatable relative to the rail along a longitudinal dimension of the rail. The manipulator can include an arm, a wrist coupled to the arm, an instrument holding portion configured to mount an instrument thereto, and the instrument holding portion being coupled to the arm by the wrist. In a mounted state of an instrument to the instrument holding portion, a shaft of the instrument extends along a first axis. The wrist can include a first degree of freedom of motion providing for rotation of the instrument holding portion about a second axis and a second degree of freedom of motion providing for rotation of the instrument holding portion about a third axis, wherein the second and third axes are offset from the first axis.

In accordance with at least one embodiment of the present disclosure, a teleoperable manipulator system can include a table assembly, a rail coupled to the table assembly, and a manipulator coupled to the rail. The table assembly can include a platform configured to support a body, the platform having lateral and longitudinal dimensions. The manipulator can be translatable relative to the rail along a longitudinal dimension of the rail. The manipulator can include a distal arm coupled to an intermediate member for rotation of the distal arm relative to the intermediate member, a wrist coupled to a distal portion of the distal arm, and an instrument holding portion configured to mount an instrument thereto. The instrument holding portion can be coupled to the wrist for rotation of the instrument holding portion relative to the distal arm by a pitch axis and a yaw axis orthogonal to the pitch axis, the pitch axis and the yaw axis being axes of the wrist.

In accordance with at least one embodiment of the present disclosure, a teleoperable manipulator system can include a table assembly, a rail coupled to the table assembly, and a manipulator coupled to the rail. The table assembly can include a platform configured to support a body, and the platform can include lateral and longitudinal dimensions. The manipulator can be translatable relative to the rail along a longitudinal dimension of the rail. The manipulator can include a proximal arm coupled to the rail via one or more proximal joints, and an intermediate arm coupled to the proximal arm via an intermediate joint. The proximal arm can have an asymmetrical shape such that, in extending between a proximal end portion of the proximal arm and a distal end portion of the proximal arm, a centerline of the proximal arm deviates from a straight line extending between the proximal end portion of the proximal arm and the distal end portion of the proximal arm.

In accordance with at least one embodiment of the present disclosure, a teleoperable manipulator system, can include a table assembly, a rail coupled to the table assembly, and a manipulator coupled to the rail. The table assembly can include a platform configured to support a body, and the platform can include lateral and longitudinal dimensions. The manipulator can be translatable relative to the rail along a longitudinal dimension of the rail. The manipulator can include a proximal arm coupled to the rail, an intermediate arm coupled to the proximal arm, and a distal arm coupled to the proximal arm. The manipulator can be configured to be stowed beneath the platform and, in a stowed state of the manipulator, the distal arm and the intermediate arm can be parallel to one another and overlap along the lateral dimension and the distal arm the proximal arm overlap along the height dimension.

In accordance with at least one embodiment of the present disclosure, a teleoperable manipulator system can include a table assembly, a rail coupled to the table assembly, and a manipulator coupled to the rail. The table assembly can include a platform configured to support a body, and the platform can include lateral and longitudinal dimensions. The manipulator can be translatable relative to the rail along a longitudinal dimension of the rail. The manipulator can include a proximal arm coupled to the rail via a proximal joint housing, an intermediate arm coupled to the proximal arm, and a distal arm coupled to the proximal arm. The manipulator can be configured to be deployable in a first configuration in which the proximal arm is in a concave orientation relative to the platform and the proximal joint housing extends outward from the rail and in a second configuration in which the proximal arm is in a convex orientation relative to the platform and the proximal joint housing extends inwards from the rail.

In accordance with at least one embodiment of the present disclosure, a teleoperable manipulator system can include a table assembly, a rail coupled to the table assembly, and a manipulator coupled to the rail. The table assembly can include a platform configured to support a body, and the platform can include lateral and longitudinal dimensions. The manipulator can be translatable relative to the rail along a longitudinal dimension of the rail. The manipulator can include a proximal arm coupled to the rail via one or more proximal joints, and an intermediate arm coupled to the proximal arm via an intermediate joint. The proximal arm can follow a non-straight path between the proximal joints and the intermediate joint.

### BRIEF DESCRIPTION OF THE DRAWINGS

The present disclosure can be understood from the following detailed description, either alone or together with the accompanying drawings. The drawings are included to provide a further understanding of the present disclosure and are incorporated in and constitute a part of this specification. The drawings illustrate one or more embodiments of the present teachings and together with the description explain certain principles and operation. In the drawings:

FIG. 1 is a schematic side view of an embodiment of a table-mounted manipulator system in a first state.

FIG. 2 is a perspective view of another embodiment of a table-mounted manipulator system.

FIG. 3 is a perspective view of an embodiment of a first manipulator of the table-mounted manipulator system of FIG. 2.

FIG. 4 is a perspective view of an embodiment of a second manipulator of the table-mounted manipulator system of FIG. 2.

FIG. 5 is a perspective view of the table-mounted manipulator system of FIG. 2 with manipulators in a first state.

FIG. 6A is a perspective view of the table-mounted manipulator system of FIG. 2 with manipulators in a second state.

FIG. 6B is a perspective view of the table-mounted manipulator system of FIG. 2 with manipulators in a third state.

FIG. 7 is a perspective view of the table-mounted manipulator system of FIG. 2 with manipulators in a fourth state.

FIG. 8 is another perspective view of a portion of the table-mounted manipulator system of FIG. 2 with manipulators in the fourth state.

FIG. 9A is a schematic perspective view of the table-mounted manipulator system of FIG. 2 with manipulators in a fifth state.

FIG. 9B is a schematic perspective view of the table-mounted manipulator system of FIG. 2 with manipulators in a sixth state.

FIG. 10 is a perspective view of the table-mounted manipulator system of FIG. 2 with manipulators in a seventh state.

FIG. 11 is a perspective view of the table-mounted manipulator system of FIG. 2 with manipulators in an eighth state.

FIG. 12A is a schematic perspective view of an embodiment of a proximal link assembly in a first state.

FIG. 12B is a schematic perspective view of the proximal link assembly of FIG. 12A a second state.

FIG. 13 is an exploded perspective view of portions of the proximal link assembly of FIG. 12A.

FIG. 14 is a schematic perspective view of an embodiment of a wrist of a manipulator.

FIG. 15 is a front view of another embodiment of a table-mounted manipulator system with manipulators in deployed states.

FIG. 16 is a perspective view of one of the manipulators of the table-mounted manipulator system of FIG. 15.

FIG. 17 is a side view of an embodiment of the proximal link assembly of the manipulator of FIG. 16.

FIG. 18 is another side view of the proximal link assembly of FIG. 17.

FIG. 19 is a side view of another embodiment of a proximal link assembly.

FIG. 20 is a side view of the table-mounted manipulator system of FIG. 15 with the manipulators in a stowed state.

FIG. 21 is a front view of the table-mounted manipulator system of FIG. 15 with the manipulators in the stowed state.

FIG. 22 is a top view of one of the manipulators of the table-mounted manipulator system of FIG. 15 in the stowed state.

FIG. 23 is a perspective view of one of the manipulators of the table-mounted manipulator system of FIG. 15 in the stowed state.

FIG. 24 is a side view of one of the manipulators of the table-mounted manipulator system of FIG. 15 in the stowed state.

FIG. 25 is a side view of another embodiment of a manipulator that can be used in the table-mounted manipulator system of FIG. 1, with the manipulator in the stowed state.

FIG. 26 is a side view of another embodiment of a manipulator that can be used in the table-mounted manipulator system of FIG. 1, with the manipulator in the stowed state.

FIG. 27 is a side view of another embodiment of a manipulator that can be used in the table-mounted manipulator system of FIG. 1, with the manipulator in the stowed state.

### DETAILED DESCRIPTION

As noted above, there can be certain challenges arising from having manipulators attached to a table assembly in a table-mounted manipulator system, such as challenges associated with constraints on the positioning the manipulators.

To address challenges with table-mounted manipulator systems, various embodiments disclosed herein contemplate a table-mounted manipulator system including a table assembly, a rail that is coupled to the table assembly, and one or more manipulators coupled to the rail. The table assembly includes a platform to support a patient or other workpiece. The manipulators are translatable relative to the table assembly along the rail. In some embodiments, the rail is also optionally translatable relative to the table assembly. The translation of the manipulators along the rail (combined with the translation of the rail in some embodiments) allows for a relatively wide range of motion of the manipulators along a longitudinal dimension of the table assembly. The wide range of motion can beneficially allow the manipulators to reach a variety of locations along the patient, and also can facilitate moving the manipulators more fully out of the way when desired.

Furthermore, in embodiments disclosed herein, the manipulators include a proximal link assembly, a distal link assembly, and an intermediate link assembly coupled in series by joints. (In some embodiments, the intermediate link assembly is omitted and the proximal and distal link assemblies are coupled directly together, and in other embodiments multiple intermediate link assemblies can be coupled in series between the proximal and distal link assemblies.) The proximal link assembly can include a proximal arm that is rotatably coupled to the rail via one or more proximal joints. For example, the proximal link assembly can include a first proximal joint coupled between the rail and a proximal end of the proximal arm, with the first proximal joint providing for rotation of the proximal arm relative to the rail around a first axis that is perpendicular to a longitudinal dimension of the platform in a neutral position of the table assembly.

In some embodiments, the proximal link assembly of certain of the manipulators further includes a second proximal joint coupled between the rail and the first joint, with the second proximal joint providing for rotation of the first joint (and hence the proximal arm coupled thereto) relative to the rail around a second axis orthogonal to the first axis and parallel to a longitudinal dimension of the rail. For example, the second axis can be horizontal when the table assembly is in a neutral position. Rotation of the proximal arm around this second proximal joint causes the proximal arm to incline or decline relative to the horizontal plane, thus raising or lowering a distal end of the proximal arm relative to the rail (and hence raising or lowering more distal portions of the manipulator, which are coupled to the distal end of the proximal arm). In addition, as the proximal arm inclines relative to the horizontal plane, movement of the proximal arm can cause more distal portions of the manipulator to correspondingly both raise and extend further across the table (as opposed to vertical movement alone). The ability to incline and/or decline the proximal arm can increase the range of motion of the overall manipulator and allow positions and poses of the manipulator to be achieved that might otherwise be difficult or not possible, as described in greater detail below.

In some embodiments, the proximal arm is extendable and retractable. For example, the proximal arm can include two or more links (for example, coaxially nested links in some embodiments), that are translatable relative to one another in a telescoping fashion to extend or retract the proximal arm. The extendibility of the proximal arm allows for an increased range of motion (e.g., lengthening) of the manipulator and allows positions and poses of the manipulator to be achieved that might otherwise be difficult or not possible, as described in greater detail below.

In some embodiments, the proximal arm has an asymmetrical shape, meaning that, while extending from a proximal end portion of the proximal arm to a distal end portion of the proximal arm, the proximal arm follows a non-straight path, i.e., a path that deviates from a hypothetical straight line extending between (i.e., connecting) the two end portions. For example, in some embodiments the proximal arm has a smoothly curved shape (e.g., an arced shape), while in other embodiments the proximal arm has a segmented shape including multiple straight and/or curved segments joined together at angles (e.g., an L-shape).

In some examples, the asymmetrical shape of the proximal arm results in the proximal arm having a concave side and a convex side, and the concave side defines an open space adjacent thereto. This open space would otherwise have been occupied by the proximal arm if the proximal arm were to extend in a straight line between the proximal and intermediate joints, but because the proximal arm is asymmetrical the open space is not occupied by the proximal arm. This open space can allow the manipulator to be placed in poses that otherwise would not be possible with a straight proximal arm of similar size. For example, if the proximal arm is rotated upwards and towards the table, a straight proximal arm will need to be stopped at a given point to avoid a collision between the arm and the patient, the table, or other objects, whereas one of the asymmetrical proximal arms disclosed herein can be able to continue rotating some distance beyond that given point because the patient, table, or other object, which would have otherwise collided with the straight arm, can instead fit within the open space along the concave side of the arm. This additional range of rotation of the proximal arm can allow, for example, for the manipulator to reach farther across the table. As another example, when the manipulators are stowed, the open space provided along the concave side of the proximal arm can allow for a more compact pose of the manipulator, as one of the links of the manipulator can be positioned at least partially within the open space. For example, in some embodiments, in the stowed state the distal arm and the proximal arm are both positioned on the same laterally extending side of the intermediate arm as one another, the distal arm is positioned directly below the proximal arm, and the distal arm is parallel to the intermediate arm. This compact pose is enabled because the distal arm can extend into the open space formed along the concave side of the proximal arm.

In some embodiments with asymmetrically shaped proximal arms, the manipulators can be selectively deployable in two different configurations, including a first configuration in which the proximal arm is in a concave orientation relative to the platform and a second configuration in which the proximal arm is in a convex orientation relative to the platform. These different configurations of the manipulator can allow the manipulators to reach a greater variety of poses, as some poses that might not be difficult or not possible with one configuration can be possible or easier with the other, and vice versa. For example, in the first configuration of the manipulator (with the concave orientation of the proximal arm), the manipulator can be able to reach farther across the platform than would be possible in the second configuration. As another example, in the second configuration of the manipulator (with the convex orientation of the proximal arm), the manipulator can be able to pitch the instrument holding portion farther backward relative to a distal link when utilizing entry ports that are located low on a patient.

In some embodiments disclosed herein, the intermediate link assembly includes an intermediate arm. In some embodiments, the intermediate arm is extendable and retractable. For example, the intermediate arm can include two or more links (for example, coaxially nested links in some embodiments), that are translatable relative to one another in a telescoping fashion to extend or retract the intermediate arm. In some embodiments, the intermediate arm can be rotatably coupled to the distal end portion of the proximal arm via one or more intermediate rotary joints. For example, a first intermediate joint can be coupled between the proximal arm and the intermediate arm, with the first intermediate joint providing for rotation of the intermediate arm relative to the proximal arm about a third axis perpendicular to the intermediate arm and the proximal arm. In some embodiments, a distal end of the intermediate arm is rotatable relative to the proximal arm about an axis that is parallel to a longitudinal dimension of the intermediate arm. For example, in some embodiments in which the intermediate arm includes two or more translatable links, the links can also be rotatable relative to one another. The extendibility of the intermediate arm allows for an increased range of motion of the manipulator and allows positions and poses of the manipulator to be achieved that might otherwise be difficult or not possible, as will be described in greater detail below.

Various aspects of embodiments of table-mounted manipulator systems described above and further described below increase the range of motion of the manipulators, and these aspects can be included individually in some embodiments or in various combinations in other embodiments (including all of the aspects being included together in some embodiments). Each of these aspects can individually contribute to increasing the range of motion of the manipulators, and in embodiments in which multiple of these aspects are combined, the effect can be even greater. For example, the ability to raise and lower the distal end portion of the proximal arm (via rotation of the proximal arm about the second proximal joint), the ability to extend and retract the proximal arm, and/or the ability to extend and retract the intermediate arm can, individually or collectively (in embodiments in which multiple of the aforementioned aspects are combined) allow a manipulator to reach locations that are far from the mounting point of the manipulator, while also being able to reach locations that are close to a mounting point of the manipulator. For example, in some embodiments the aspects described above and further below can allow a manipulator to extend over the patient and across the platform so that a distal end portion of the manipulator is adjacent to the side of platform opposite from the side the manipulator is mounted to. This can, for example, allow an instrument mounted to the manipulator to use an entry port that is located on a side of the patient that is opposite from the side of the platform that the manipulator is mounted to. This ability of certain manipulators in embodiments disclosed herein to reach across the platform to an opposite side can allow for fewer manipulators to be provided along a given side of the table while maintaining suitability of the system for procedures that can normally require more manipulators along the given side. For example, a procedure that uses three entry ports disposed along one side of a patient can conventionally need three manipulators to be positioned on the side of the table assembly that is closest to the three entry ports, whereas in some embodiments disclosed herein, a system with two manipulators provided on the side of the table closest to the entry ports and at least one manipulator provided on the opposite side of the table could perform the same procedure (e.g., with the manipulator on the opposite side of the table assembly extending over and across the platform to reach one of the entry ports). By reducing the number of manipulators disposed on a given side of the platform, embodiments disclosed herein make it easier to move the manipulators out of the way for certain tasks that require space along the longitudinally extending sides of the table (e.g., transferring a patient from a gurney to the table), as it can be more difficult, for example, to move three manipulators out of the way when they are all attached to the same rail than it would be to move four manipulators out of the way when two are attached to one rail and two are attached to another rail.

Moreover, the ability of certain manipulators to reach locations on both longitudinally extending sides of the platform allows a single system with a given arrangement of manipulators to be used in a variety of procedures, including procedures with a variety of different entry port arrangements. This is in contrast to using a specialized system for each type of entry port arrangement. For example, an embodiment disclosed herein that has two manipulators coupled to one side of the platform and two manipulators coupled to the other side of the platform can be used with both the above-described port arrangement of three entry ports disposed along one side of the patient as well as other port arrangements such as ones in which four ports are disposed in a laterally extending line along a middle portion of the patient. In contrast, for a conventional system to be able to accommodate both of these types of port arrangements, the system can need to have at least three manipulators coupled to one side of the platform and at least two coupled to the other side. Thus, systems disclosed herein can allow for a greater variety in port placement and/or a reduction in the number of manipulators.

Moreover, in some embodiments, the distal link assembly includes a distal arm, an instrument holding portion, and a wrist for movably coupling the distal arm to the instrument holding portion. In some embodiments, the wrist provides three orthogonal rotational degrees of freedom of motion for the instrument holding portion relative to the distal arm. The degrees of freedom of motion provided by the wrist can allow the manipulator to position the instrument in poses that would not otherwise be possible. For example, when reaching over the patient to an opposite side of the platform, as described above, the instrument holding portion can need to be oriented at various angles relative to the distal arm in order to position the instrument appropriately to use the entry port, and the wrist can provide the degrees of freedom of motion that facilitate such orientation of the instrument holding portion. In some embodiments, some of the degrees of freedom of motion of the wrist mechanism are driven by actuators disposed remotely from the wrist, such as in the distal arm, with actuation elements such as cables extending from the actuators to the wrist to drive the motion of the wrist. This allows the wrist to be relatively compact while still having powered joints with sufficient strength to support and move the instrument holding portion and instrument, and can also allow the weight of the actuators to be located more proximally along the manipulator, thus reducing the moment arm (leverage) of the manipulator about the proximal joints.

### I. Table-Mounted Manipulator System

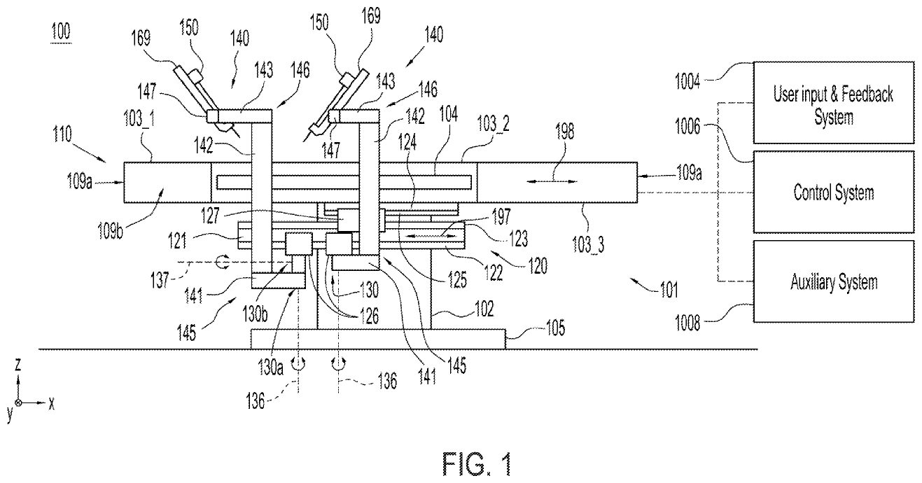

FIG. 1 illustrates an embodiment of a table-mounted manipulator system 100 (“system 100”). The system 100 includes a table assembly 101, at least one rail assembly 120 coupled to the table assembly, and one or more manipulators 140 coupled to each rail assembly 120. Each manipulator 140 can carry one or more instruments 150, which can be removably or permanently mounted thereon. As shown in FIG. 1, the system 100 also can include a control system 1006, a user input and feedback system 1004, and/or an auxiliary system 1008. In some embodiments, the system 100 is configured as a computer-assisted, teleoperable medical system, in which case table assembly 101 can be configured to support a patient (not shown) and the instruments 150 can be medical instruments. The system 100 in this configuration can be usable, for example, to perform any of a variety of medical procedures, such as surgical procedures, diagnostic procedures, imaging procedures, therapeutic procedures, etc. Moreover, the system 100 when configured as a teleoperable medical system need not necessarily be used on a living human patient. For example, a non-human animal, a cadaver, tissue-like materials used for training purposes, and so on, can be supported on the table assembly 101 and worked on by system 100. In other embodiments, the system 100 is configured as a computer-assisted teleoperable system for use in non-medical contexts, in which case the table assembly 101 can be configured to support an inanimate workpiece (something being manufactured, repaired, tested, etc.) and the instruments 150 can be non-medical instruments, such as industrial instruments.

As shown in FIG. 1, the table assembly 101 includes a platform assembly 110 configured to support the patient or inanimate workpiece, a support column 102 coupled to and supporting the platform assembly 110, and a base 105 coupled to the support column 102. The base can be configured to contact the ground or other surface upon which the table assembly 101 rests to provide stability for the table assembly 101. In some embodiments, the base 105 is omitted. In some embodiments, the base 105 includes mobility features, such as wheels, tracks, or other such features (not shown), to allow movement of the table assembly 101 along the ground or other surface. In FIGS. 1, the support column 102 is illustrated as a single vertical columnar part to simplify the discussion, but the support column 102 could take any desired shape and could include any number of parts. For example, the support column 102 can include horizontal support structures (not illustrated) such as beams, rails, etc. to couple the platform assembly 110 to a vertical portion of the support column 102. Moreover, in various embodiments, the support column 102 can be telescoping and configured to extend and contract in height.

The platform assembly 110 includes one or more platform sections 103 to support the patient or workpiece. The platform sections 103 each have a support surface configured to contact and support the patient or workpiece. In some embodiments multiple platform sections 103 are used and the platform sections 103 are arranged in series to support different portions of the patient or workpiece. For example, in the embodiment illustrated in FIG. 1, the platform assembly 110 includes a first end section 103_1, one or more middle sections 103_2, and a second end section 103_3, with the one or more middle sections 103_2 being arranged between the two end sections 103_1 and 103_3. In some embodiments, the first end section 103_1 can be configured to support a head of the patient, the second end section 103_3 can be configured to support the feet and/or legs of the patient, and the one or more middle sections 103_2 can be configured to support a torso and/or other portions of the patient. For convenience, the side of the platform assembly 110 that is near the first end section 103_1 (e.g., a left side in the orientation shown in FIG. 1) will be referred to herein as a “head” of the platform assembly 110 (or “head side” or “head end”) and the side of the platform assembly 110 that is near the second end section 103_3 (e.g., a right side in the orientation shown in FIG. 1) will be referred to herein as a “foot” of the platform assembly 110 (or “foot side” or “foot end”), but this is merely an arbitrary convention chosen herein for convenience of description and is not intended to limit the configuration or usage of the table assembly 101 (e.g., a head of a patient could be positioned at the “foot” side of the platform assembly 110 if desired, and vice versa). The relative positions of two components or of two portions of a single component can also be described using “head” and “foot” (e.g., a “head end” and a “foot end” of a rail 121) with “head” referring to the component or portion that is relatively closer to the head end of the table assembly 110 and “foot” referring to the component or portion that is relative closer to the foot end of the table assembly 110. In other embodiments, different numbers and arrangements of platform sections 103 are used, including one, two, four, or more platform sections 103. In some embodiments, one or more of the platform sections 103 can be movable relative to other platform sections 103 and/or relative to the support column 102. For example, in some embodiments, some or all of the platform sections 103 are coupled to adjacent platform sections 103 and/or to the support column 102 by rotatable joints such that at least some of the platform sections 103 can tilt relative to one another and/or relative to the support column 102. The platform assembly 110 can also be movable as a whole relative to the support column 102, as described in greater detail below.

The platform assembly 110 has a longitudinal dimension 198 (e.g., parallel to the x-axis in FIG. 1), a lateral dimension orthogonal to the longitudinal dimension (e.g., parallel to the y-axis in FIG. 1), and a thickness or height dimension orthogonal to both the longitudinal dimension 198 and lateral dimension (e.g., parallel to the z-axis in FIG. 1). As used herein, the longitudinal dimension 198 refers to a dimension of greatest extent of the platform assembly 110 when all of the platform sections 103 of the platform assembly are fully extended and all are oriented with their support surfaces roughly aligned in a same plane with one another (or when as close to this state as possible) so as to collectively form a combined support surface that is substantially planar with potentially small gaps between adjacent platform sections 103. In general, the longitudinal and lateral dimensions of the platform assembly 110 and the support surfaces of the platform sections 103 are oriented roughly parallel to the ground or other surface on which the table assembly 101 is supported when the platform assembly 110 is in a neutral configuration. However, one of ordinary skill in the art would understand that the platform assembly 110 as a whole and/or individual platform sections 103 thereof do not necessarily have to be parallel to the ground, and that one or both of the longitudinal and/or lateral dimensions can be tilted relative to the ground in various configurations through which the platform assembly 110 and/or platform section 103 can be movable, including in a neutral configuration in some cases. The platform assembly 110 and the various platform sections 103 thereof have various sides or faces that extend along the longitudinal dimension 198 or lateral dimension, and these can be referred to herein as longitudinally extending sides (or faces) and laterally extending sides (or faces), respectively. Specifically, a longitudinally extending side (or face) is a side (or face) of the platform assembly 110 or of a platform section 103 that extends along the longitudinal dimension 198 of the platform assembly 110 (i.e., along an x-direction in FIG. 1). For example, one longitudinally extending side 109b of the platform assembly 1110 is indicated in FIG. 1. Similarly, a laterally extending side (or face) is a side (or face) of the platform assembly 110 or of a platform section 103 that extends along the lateral dimension of the platform assembly 110 (i.e., along a y-direction in FIG. 1). For example, two laterally extending sides 109a of the platform assembly 110 are indicated in FIG. 1.

At least one of the platform sections 103 is directly coupled to and supported by the support column 102. The remaining platform sections 103 can be coupled directly to the support column 102 or they can be coupled indirectly to the support column 102 via a chain of one or more intervening platform sections 103. For example, in some embodiments a main platform section 103 (e.g., a middle section 103_2) is coupled to and directly supported by the support column 102 and the others of the platform sections 103 (e.g., end sections 103_1 and 103_3) are coupled to the main platform section 103 or to another platform section 103. As another example, in some embodiments multiple platform sections 103 (all in some embodiments) are coupled directly to the support column 102 and not to another platform section 103.

In some embodiments, some (all, in some cases) of the above-described parts of the table assembly 101 can be movable relative to one another. For example, in some embodiments the platform assembly 110 as a whole can be moved relative to the support column 102, such as by tilting around a horizontal axis, swiveling around a vertical axis, translating vertically along the support column 102, translating horizontally relative to the support column 102, and so on. In some embodiments, such movement of the platform assembly 110 as a whole can be provided by one or more joints that couple a main platform section 103 (e.g., a middle section 103_2) to the support column 102. Furthermore, as already noted above, individual platform sections 103 can be movable relative to one another and relative to the support column 102 as well, which can be facilitated by joints coupling the platform sections 103 to the support column 102 or to adjacent platform sections 103.

In some embodiments, the platform assembly 110 also includes one or more accessory rails 104. The accessory rails 104 can be configured to receive accessory devices removably mounted thereon, such as such as leg stirrups, liver retractor, arm boards, and bed extenders. In some embodiments, the accessory rails 104 adhere to industry standard specifications familiar to those of ordinary skill in the art to allow compatibility with accessory devices compliant with the standard. The accessory rails 104 can be attached to longitudinally extending side faces of one or more of the platform sections 103. One or more openings can be defined between an accessory rail 104 and the side face of the platform section 103 to which the accessory rail 104 is attached and portions of accessories mounted to the accessory rail 104 can be inserted through the openings.

As noted above, the system 100 also includes one or more manipulators 140. FIG. 1 illustrates two manipulators 140, but any number of manipulators 140 can be included (such as, for example, one, two, three, or more manipulators mounted to each rail assembly 120, as described in further detail below). A manipulator 140 can include a kinematic structure of links coupled together by one or more joints. Specifically, the manipulators 140 each include a proximal link assembly including a proximal arm 141 movably coupled to the rail assembly 120 via one or more proximal joints 130, an intermediate link assembly including an intermediate arm 142 movably coupled to the proximal link assembly via one or more intermediate joints 145, and a distal link assembly including a distal arm 143 movably coupled to the intermediate link assembly by one or more distal joints 146. The distal link assembly can also include an instrument holding portion 169 coupled to the distal arm 143 and configured to carry the instrument 150.

The manipulator 140 is movable through various degrees of freedom of motion provided by various joints, including the proximal, intermediate, and distal joints 130, 145, and 146, thus allowing an instrument 150 mounted thereon to be moved relative to the worksite. Some of the joints can provide for rotation of links relative to one another, other joints can provide for translation of links relative to one another, and some can provide for both rotation and translation. In particular, in some embodiments, the proximal arm 141 is rotatably coupled to the rail 121 via a first proximal joint 130a, which provides for rotation of the proximal arm 141 relative to the rail 121 around a first axis 136 that is perpendicular to a longitudinal dimension 197 of the rail 121 (e.g., perpendicular to the x-direction in FIG. 1). In a neutral state of the proximal arm 141, the first axis 136 is also perpendicular to a lateral dimension of the rail 121 (e.g., perpendicular to the y-direction in FIG. 1), and thus in this state the first axis 136 is oriented vertically (i.e., perpendicular to the aforementioned horizontal plane, or in other words oriented in the z-direction in FIG. 1). In addition, in a neutral state of the table assembly 101, in which the platform 110 is parallel to the ground and the rail 121 (i.e., an x-direction in the orientation of FIG. 1), the first axis 136 is also perpendicular to the longitudinal axis 198 of the platform 110, but this is not necessarily the case in other states (e.g., states in in which the platform 110 is tilted relative to the rail 121, which can be possible in some embodiments).

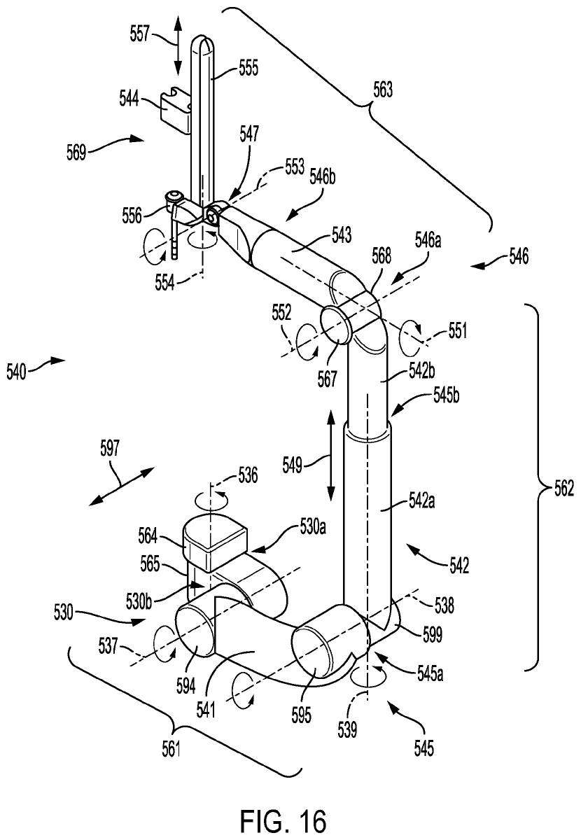

In some embodiments, the proximal link assembly of certain of the manipulators 140 is configured to allow for rotation of the proximal link 141 about a second axis 137, in addition to allowing for rotation about the first axis 136, with the second axis 137 being orthogonal to the first axis 136. In some embodiments, the rotation about the second axis 137 can be provided by a second proximal joint 130b included in the proximal link assembly. In particular, in some embodiments the proximal link assembly of certain of the manipulators 140 further includes a second proximal joint 130b, and the first and second proximal joints 130a and 130b together couple the proximal arm 141 to the rail 121, with the second proximal joint 130b providing for rotation of the proximal arm 141 relative to the rail 121 around a second axis 137 orthogonal to the first axis 136 and parallel to a longitudinal dimension 197 of the rail 121 (e.g., x-direction in FIG. 1). In some embodiments, the second proximal joint 130b is coupled between the rail 121 and the first proximal joint 130a, while in other embodiments the second proximal joint 130b is coupled between the first proximal joint 130a and the proximal 141 (not shown in FIG. 1) (see, for example, FIG. 16 which illustrates one embodiment in which the second proximal joint 530b is coupled between first proximal joint 530a and proximal arm 541). In still other embodiments, the rotation about the second axis 137 is provided by the first proximal joint 130a without the addition of a second proximal joint (e.g., the first proximal joint 130a is configured to provide rotation about multiple axes, such as a ball-and-socket joint). The longitudinal dimension 197, and hence the second axis 137, is parallel to the ground in some embodiments. In the neutral state of the table assembly 101, the second axis 137 is also parallel to the longitudinal axis 198 of the platform 110, but this is not necessarily the case in other states (e.g., states in in which the platform 110 is tilted relative to the rail 121, which can be possible in some embodiments). Rotation of the proximal arm 141 around this second axis 137 (e.g., via the second proximal joint 130b) causes the proximal arm 141 to incline or decline relative to the horizontal plane, thus raising or lowering a distal end of the proximal arm 141 relative to the rail 121. In addition, as the proximal arm 141 inclines relative to the horizontal plane, movement of the proximal arm 141 can cause more distal portions of the manipulator 140 to correspondingly both raise and extend further across the table (as opposed to vertical movement alone). In some embodiments, the rotation about the second axis 137 (e.g., via second proximal joint 130b) allows the proximal arm 141 to be moved between orientations ranging at least between a horizontal orientation and a vertical inclined orientation (e.g., at least 90 degrees of rotation). In some embodiments, rotation about the second axis 137 (e.g., via the second proximal joint 130b) can also allow for rotation of the proximal arm 141 to orientations that are declined relative to a horizonal orientation. In some embodiments, certain manipulators 140 are provided with the ability to rotate about the second axis 137 (e.g., via the second proximal joint 130b) while others are not. For example, in some embodiments a first manipulator 140 whose proximal arm 141 is positionable under a second manipulator 140 in a nested configuration (described further below) can be provided with the second proximal joint 130b (e.g., because the lower positioning of the proximal arm 141 makes room for the proximal joint 130b), while a second proximal joint 130b can be omitted in the second manipulator 140 (e.g., because the higher positioning of the proximal arm 141 of the second manipulator 140 does not leave sufficient room for the second joint). In other embodiments (not illustrated), coupled to a same rail 121 all of the manipulators 140 (or all manipulators 140 in the system 100, in some embodiments) are provided with the ability to rotate about the second axis 137 (e.g., via second proximal joints 130b). In still other embodiments (not illustrated), none of the manipulators 140 coupled to a given rail 121 (or none of the manipulators 140 in the entire system 100, in some embodiments) are provided with the ability to rotate about the second axis 137.

In addition, in some embodiments, the proximal arm 141 is extendable and retractable. For example, the proximal arm 141 can include two or more links that are translatable relative to one another in a telescoping fashion to extend or retract the proximal arm 141. In other words, these two or more links are coupled together by, or they themselves form, a prismatic joint. For example, in some embodiments the proximal arm 141 includes an outer link that has a bore (for example an axial bore extending along a longitudinal axis of the proximal arm 141) and an inner link that is nested within the outer link in the bore thereof.

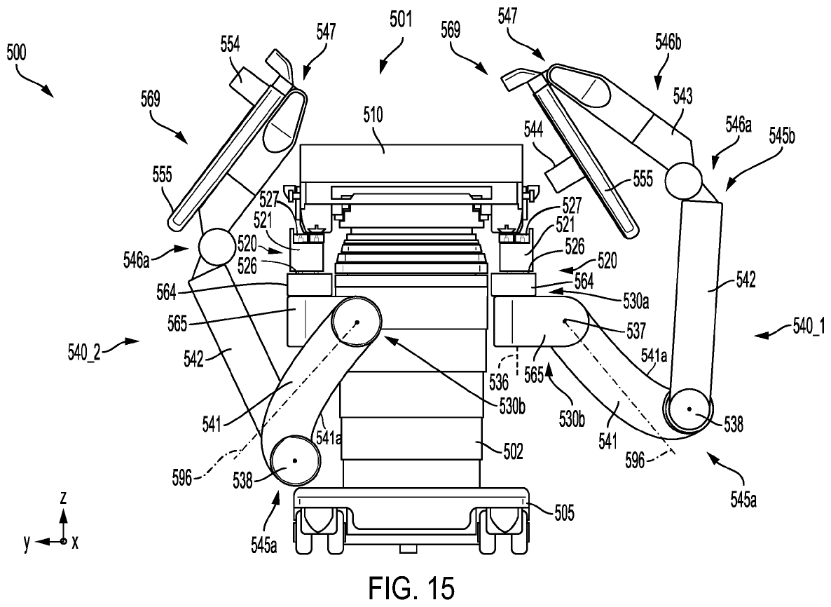

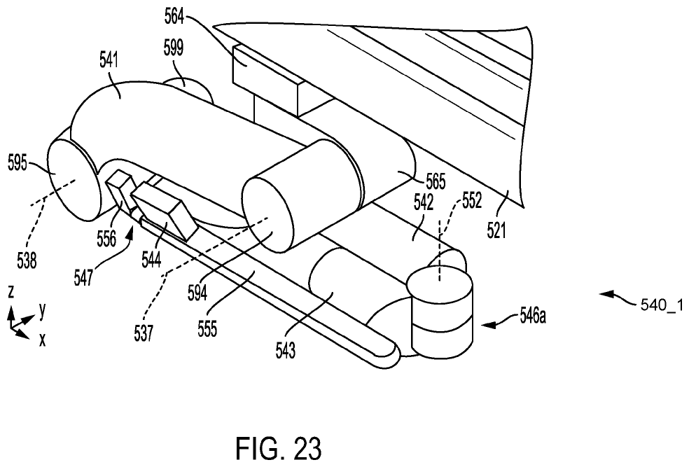

In addition, in some embodiments, the proximal arm 141 has an asymmetrical shape, meaning that in extending from a proximal end portion of the proximal arm 141 to a distal end portion of the proximal arm 141, the proximal arm 141 follows a non-straight path, i.e., a path that deviates from a hypothetical straight line extending between (i.e., connecting) the two end portions. More specifically, the proximal arm 141 can extend between the proximal joint 130 coupled to the proximal end portion of the proximal arm 141 and an intermediate joint 145 (described below) coupling the distal end portion of the proximal arm 141 to intermediate arm 142, with a centerline of the proximal arm 141 extending between these joints 130 and 145 deviating from a straight line between respective axes of the joints 130 and 145. For example, in some embodiments the proximal arm 141 has a smoothly curved shape (e.g., a centerline of the proximal arm follows a smoothly curved path), while in other embodiments the proximal arm 141 has a segmented shape including multiple straight and/or curved segments joined together at angles (e.g., an L-shape). Various embodiments of a proximal arm with an asymmetrical shape, which can be configurations of the proximal arm 141, are described in greater detail below with reference to FIGS. 15-24.

In some embodiments, the intermediate arm 142 can be rotatably coupled to the distal end portion of the proximal arm 141 via one or more intermediate rotary joints 145. For example, the intermediate joints 145 can provide for rotation of the intermediate arm 142 relative to the proximal arm 141 about a third axis (not illustrated) perpendicular to the intermediate arm 142 and the proximal arm 141. In addition, in some embodiments, the intermediate joints 145 can provide for rotation of a distal end of the intermediate arm 142 relative to the proximal arm 141 about an axis that is parallel to a longitudinal dimension of the intermediate arm 142. In some embodiments, the intermediate arm 142 is also extendable and retractable. For example, the intermediate arm 142 can include two or more links that are translatable relative to one another in a telescoping fashion to extend or retract the intermediate arm 142, in a manner similar to that described above in relation to proximal arm 141. In some embodiments, the links of the intermediate arm 142 are both translatable relative to one another along a longitudinal dimension of the intermediate arm 142 and also rotatable relative to one another about an axis parallel to the longitudinal dimension of the intermediate arm 142, thus providing for the above-described rotation of the distal end of the intermediate arm 142 relative to the proximal arm 141 about the axis that is parallel to a longitudinal dimension of the intermediate arm 142.

Moreover, in some embodiments, the distal arm 143 is movably coupled to the instrument holding portion 169 via a wrist 147, which includes joints for moving the instrument holding portion 169 relative to the distal arm 143. The joints of the wrist 147 can be referred to herein as wrist joints. In some embodiments, the wrist 147 provides multiple rotational degrees of freedom motion. For example, in some embodiments the wrist 147 has three rotational degrees of freedom of motion for the instrument holding portion 169 relative to the distal arm 143. For example, the wrist 147 can be rotatably coupled to the distal arm 143 to provide a roll degree of freedom of motion including rotation of the wrist 147 as a whole about an axis parallel to the distal arm 143, and the wrist 147 can further include two joints for providing yaw and pitch degrees of freedom of motion including rotation around pitch and yaw axes which are perpendicular to one another. One of the pitch and yaw axes is also perpendicular to the roll axis (the other of the pitch and yaw axes can also be perpendicular to the roll axis in a neutral state of the wrist 147, but not necessarily in other states). In some embodiments, the joints providing some of the degrees of freedom of motion of the wrist 147 (e.g., yaw and pitch, in some embodiments) are driven by actuators disposed remotely from the wrist 147, such as in a more proximal portion of the manipulator 140 with actuation elements (such as cables, filaments, belts, bands, linkages, etc.) extending from the actuators to the wrist 147 to drive the motion of the wrist. For example, in some embodiments, the wrist includes two wrist joints disposed in the wrist that provide rotation about the yaw and pitch axes, and these two wrist joints can be coupled to actuation elements (e.g., cables) that drive the rotation. In some embodiments, the actuators that drive the wrist 147 are positioned in the distal arm 143. Disposing the actuators remotely from the wrist 147 allows the wrist 147 to be more compact. Wrists that are compact, such as the wrists 147, can be positioned more closely to portions of other manipulators 140, in some circumstances, which can allow for greater flexibility in the positioning and posing of the manipulators 140. Moreover, placing the actuators in a more proximal portion of the manipulators 140, such as in the distal arm 143, moves the weight of the actuators closer to a proximal end of the kinematic chain that makes up the manipulator 140, thus reducing the moment arm (leverage) created by the weight of the actuators.

Some or all of the joints of the system 100 described above (as well as other joints that might be present in the system) can be powered joints, meaning a powered drive element can control movement of the joint through the supply of motive power. Such powered drive elements can include, for example, electric motors, pneumatic or hydraulic actuators, and other types of powered drive elements those having ordinary skill in the art would be familiar with. In some embodiments, the joints of the wrist 147 are powered joints. Additionally, in some embodiments some of the joints of the system 100 can be manually articulable (e.g., unpowered) joints, which can be articulated manually for example by manually moving the links coupled thereto. Joints referred to herein as unpowered can lack powered drive elements to drive articulation of the joint but still can include other powered aspects or devices, such as electronically (or hydraulically/pneumatically, etc.) controlled brakes, sensors (e.g., position, velocity, force, torque sensors), or other powered devices. Additionally, in some embodiments some of the joints of the system 100 can be partially powered and partially manually articulable—for example powered elements such as motors can assist manipulation, such as by compensating for gravity loads, but some manual force input can also be used to cause the articulation. Additionally, some joints (whether powered or not) can also be passively counterbalanced (e.g., via masses or springs). Certain joints can be actively controllable during performance of a procedure, for example, under the control of a control system 1006 in response to inputs recited at a user input and feedback system 1004. Other joints, sometimes referred to as setup joints, can be articulated during a setup phase in preparation for performance of the procedure but can generally remain more-or-less stationary during performance of the procedure. Setup joints can be powered, manually articulable, or partially powered. For example, in some embodiments, the proximal joints 130 and the prismatic joint that provides extension of the proximal arm 141 are setup joints.

As noted above, the instrument holding portion 169 is configured to support an instrument 150, and in some embodiments the instrument holding portion 169 includes a drive interface to removably couple the instrument and to provide driving inputs (e.g., mechanical forces, electrical inputs, etc.) to drive an instrument coupled thereto. For example, the drive interface can include output couplers (not illustrated) to engage (directly or indirectly via an intermediary) with input couplers (not illustrated) of the instrument 150 to provide driving forces or other inputs to the mounted instrument 150 to control various degree of freedom movement and/or other functionality of the instrument 150, such as moving an end-effector of the instrument, opening/closing jaws, driving translation and/or rotation of a variety of components of the instrument, delivery of substances and/or energy from the instrument, and various other functions those of ordinary skill in the art are familiar with. The output couplers can be driven by actuators (e.g., electrical servo-motors, hydraulic actuators, pneumatic actuators) with which those of ordinary skill in the art have familiarity. An instrument sterile adaptor (ISA) can be disposed between the instrument 150 and the instrument manipulator mount interface to maintain sterile separation between the instrument 150 and the manipulator 140. The instrument manipulator mount can also include other interfaces (not illustrated), such as electrical interfaces to provide and/or receive electrical signals to/from the instrument 150. The instruments 150 can include any tool or instrument, including for example industrial instruments and medical instruments (e.g., surgical instruments, imaging instruments, diagnostic instruments, therapeutic instruments, etc.). In some embodiments, the system 100 can include flux delivery transmission capability as well, such as, for example, to supply electricity, fluid, vacuum pressure, light, electromagnetic radiation, etc. to the end effector. In other embodiments, such flux delivery transmission can be provided to an instrument through another auxiliary system 1008, described further below and as those of ordinary skill in the art would be familiar with in the context of computer-assisted, teleoperated medical systems.

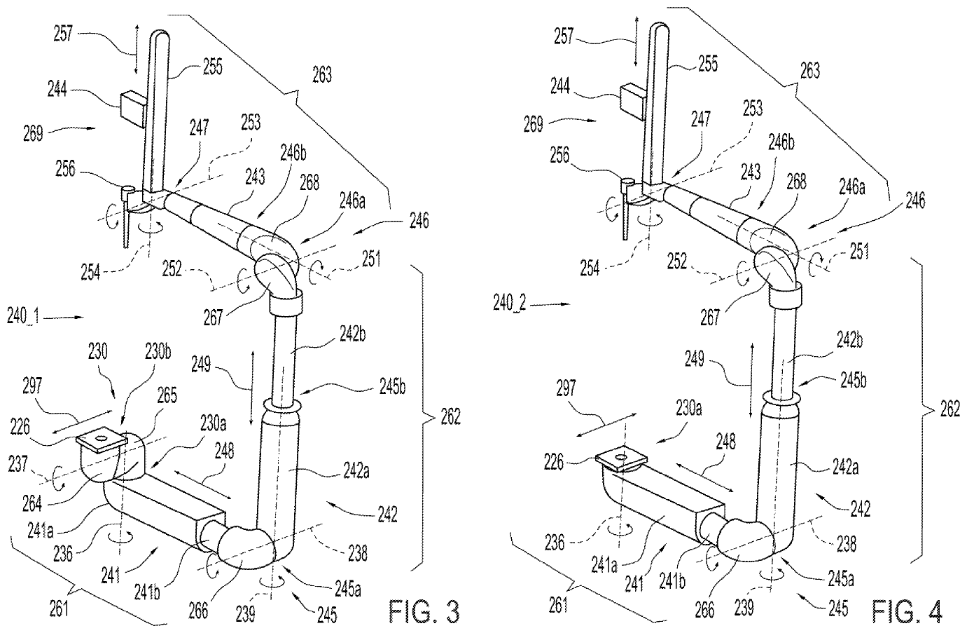

Additional details relating to the manipulators are described below with reference to FIGS. 3, 4, and 16 which illustrate various embodiments of manipulators 240 and 540 that can be used as the manipulators 140. Moreover, in some embodiments, aspects of the manipulators 140 can be similar to the manipulators described in U.S. Provisional Patent Application No. 63/336,773, entitled “RAIL ASSEMBLY FOR TABLE MOUNTED MANIPULATOR SYSTEM, AND RELATED DEVICES, SYSTEMS AND METHODS,” inventor Ryan Abbott, and in U.S. Provisional Patent Application No. 63/336,778, entitled “NESTING PROXIMAL LINKS FOR TABLE MOUNTED MANIPULATOR SYSTEM, AND RELATED DEVICES, SYSTEMS AND METHODS,” first named inventor Brain Lambrecht, both filed Apr. 29, 2022, or those described in, for example, U.S. Pat. No. 9,358,074 (filed May 31, 2013) to Schena et al., entitled “Multi-Port Surgical Robotic System Architecture,” U.S. Pat. No. 9,295,524 (filed May 31, 2013) to Schena et al., entitled “Redundant Axis and Degree of Freedom for Hardware-Constrained Remote Center Robotic Manipulator,” and U.S. Pat. No. 8,852,208 (filed Aug. 12, 2010) to Gomez et al., entitled “Surgical System Instrument Mounting,” the contents of each of which are incorporated herein by reference in their entirety. Various other embodiments of manipulators can include those as configured as part of the medical systems that are part of various da Vinci® Surgical Systems, such as the da Vinci X®, da Vinci Xi®, and da Vinci SP systems, commercialized by Intuitive Surgical, Inc., of Sunnyvale, California.

The number, locations, and types of links and joints of the manipulators, as well as the various degrees of freedom of motion thereof, are not limited to those described above. In some embodiments, manipulators include additional links, joints, and/or degrees of freedom beyond those described above. In other embodiments, manipulators can omit certain of the links, joints, and/or degrees of freedom described above. Embodiments contemplated herein include embodiments including various combinations of one or more of the links, joints, and degrees of freedom of motion described above.

As shown in FIG. 1, the manipulators 140 are coupled to the table assembly 101 via the at least one rail assembly 120. In some embodiments, multiple similar rail assemblies 120 are provided, for example one for each longitudinally extending side of the platform assembly 110. For example, in some embodiments, a first rail assembly 120 can be provided at a first longitudinally extending side of the platform assembly 110 and a second rail assembly 120 can be provided at a second longitudinally extending side of the platform assembly 110. In such embodiments with multiple rail assemblies 120, manipulators 140 can be coupled to the rail assemblies 120 in any number or combination, and because the rail assemblies 120 can be positioned along different sides of the platform assembly 110, the manipulators 140 too can be positioned along different sides of the platform assembly 110. The description below will describe one rail assembly 120 to simplify the description, but the other rail assemblies 120 (if present) can be configured similarly. The rail assembly 120 includes a rail 121 and a number of carriages 126 (also “first carriages 126”) coupled to the rail 121 and to the manipulators 140 to allow motion of the manipulators 140 along the rail 121. More specifically, the first carriage 126 can be coupled to (or can be a part of) the proximal link assembly of a corresponding manipulator 140. Each carriage 126 is moveable along a longitudinal dimension 197 of the rail 121 and couples a respectively corresponding one of the manipulators 140 to the rail 121 such that the manipulators 140 can translate relative to the rail 121 along the longitudinal dimension 197 of the rail 121. In some embodiments, the longitudinal dimension 197 of the rail 121 is parallel to the longitudinal dimension 198 of the platform assembly 110 (e.g., parallel to the x-axis) in a neutral configuration of the platform assembly 110, as shown in FIG. 1.

The movable rail 121 includes a first set of engagement features 122 configured to engage with complementary engagement features of the first carriages 126. For example, the first set of engagement features 122 of the rail 121 can include a track including flanges extending along the longitudinal dimension 197, and the complementary engagement features of the first carriages 126 are configured to engage and ride along the flanges of the first set of engagement features 122. The first set of engagement features 122 can also include a track including grooves in which the complementary engagement feature are received. Any other type of complementary engagement features that when engaged allow for relative translation can be used as the complementary engagement features, and those having ordinary skill in the art are familiar with various complementary engagement features that are used in rail and carriage systems. In some embodiments, the first set of engagement features 122 and/or the complementary engagement features can include bearing devices configured to reduce friction to facilitate easier translation, such as wheels, balls, plain bearing surfaces coated or otherwise provided with a low friction material, and other friction reducing mechanisms. In FIG. 1, one first carriage 126 is shown per manipulator 140, but multiple first carriages 126 could be provided to operably couple to and support a given manipulator 140.

In some embodiments, in addition to the manipulators 140 being movable along the rail 121, the rail 121 can also be movable relative to the table assembly 101. In such embodiments, the rail assembly 120 also includes one or more carriages 127 (also referred to as “second carriages 127”) coupled to the rail 121 and to the table assembly 101 to allow motion of the rail 121. More specifically, the carriages 127 couple the rail 121 to the table assembly 101 such that the rail 121 can translate relative to the table assembly 101 along a direction of the longitudinal dimension 197 of the rail 121. In particular, in these embodiments the rail 121 includes a second set of engagement features 123 (e.g., tracks or other engagement features) that engage with complementary engagement features of the second carriage 127 to couple the rail 121 to the second carriage 127 while allowing translation between the rail 121 and second carriage 127.

In some embodiments, the translation between the rail 121 and the table assembly 101 is provided by relative motion between the second carriages 127 and the rail 121. For example, in some embodiments the second carriages 127 are fixed relative to (e.g., are a part of) the table assembly 101 and the rail 121 and second carriages 127 are movably coupled together such that the rail 121 translates relative to the second carriages 127 along the direction of the longitudinal dimension 197 of the rail 121. One second carriage 127 is shown in FIG. 1 for ease of description, but any number could be used, including none in some embodiments.

In some embodiments, the translation of the rail 121 and the table assembly 101 is provided by relative motion between the second carriages 127 and the table assembly 101. For example, in some embodiments the second carriages 127 are fixed relative to (e.g., are a part of) the rail 121 and movably coupled to the table assembly 101 such that translation of the second carriages 127 relative to the table assembly 101 along the longitudinal dimension 197 causes the rail 121 to also translate relative to the table assembly 101.

In some embodiments, the translation between the rail 121 and the table assembly 101 is provided by a combination of relative motion between the second carriages 127 and the rail 121 and relative motion of the second carriages 127 and the table assembly 101.

In some embodiments in which the second carriages 127 are movably coupled to the table assembly 101, the rail assembly 120 further includes a second rail 124, which can be coupled between the second carriages 127 and the table assembly 101. In other embodiments the second carriages 127 can be coupled directly to the table assembly 101.

The movability of the rail 121 relative to the table assembly 101 can allow for a greater range of motion of the manipulators 140 and/or for a shortening of the rail 121, as compared to a configuration in which the rail 121 is fixed relative to the table assembly 101. This can also enable the rail assembly 120 and/or manipulators 140 to more easily be moved out of the way of the platform assembly 110 to avoid interference therewith as the platform assembly 110 and/or individual platform sections 103 thereof are moved through various configurations. However, in some embodiments the rail 121 is fixed relative to the table assembly 101 and the manipulators 140 are positioned relative to the platform assembly 110 solely through motion of the manipulators 140 along the rail 121.

In some embodiments, the rail assembly 120 is coupled to one of the platform sections 103. In other embodiments, the rail assembly 120 is coupled to the support column 102. Which structure the rail assembly 120 is coupled to can make a difference in embodiments in which the platform assembly 110 as a whole is movable relative to the support column 102, for example by tilting relative to the support column 102. In embodiments in which the rail assembly 120 is coupled to one of the platform sections 103 (e.g., a middle section 103_2), when the platform assembly 110 moves relative to the support column 102, the rail assembly 120 and hence the manipulators 140 coupled thereto move along with the platform assembly 110. This can allow the manipulators 140 to automatically maintain a set pose and position relative to the platform assembly 110, and thus relative to a patient supported on the platform assembly, regardless of a configuration of the platform assembly 110 and without having to reposition the manipulators 140. Moreover, in some circumstances, collision between the platform assembly 110 and the rail assembly 120 due to motion of the platform assembly 110 can be avoided as they both move together. In embodiments in which the rail assembly 120 is coupled to the support column, when the platform assembly 110 moves relative to the support column 102, the rail assembly 120 and hence the manipulators 140 coupled thereto remain with the support column 102 and do not move along with the platform assembly 110. This can allow greater motion of the rail assembly 120 and manipulators 140 relative to the platform assembly 110. This also can increase the strength and/or relative stiffness of the structure between the manipulators 140 and the support column 102 by reducing the length of the structure between them and reducing the number of connections between them.

In some embodiments, motors or other actuation devices (not illustrated) are provided to drive the relative translation between the rail 121 and the first carriages 126. Similarly, in embodiments in which the second carriages 127 are present, motors or other actuation devices (not illustrated) can be provided to drive the relative translation between the rail 121 and the second carriages 127 and/or between the second carriages 127 and the table assembly 101. In some embodiments, motors/actuators are housed within the rail 121. In some embodiments, motors/actuators are housed within the first and/or second carriages 126 and 127. In some embodiments, motors/actuators are housed within the table assembly 101.

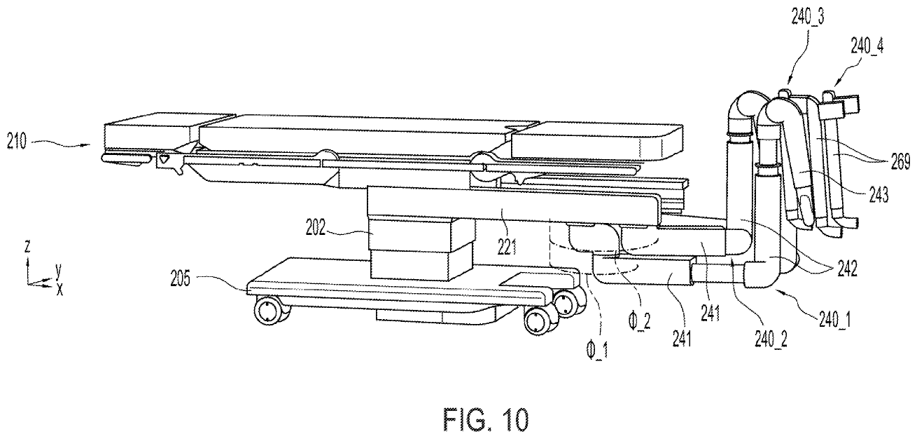

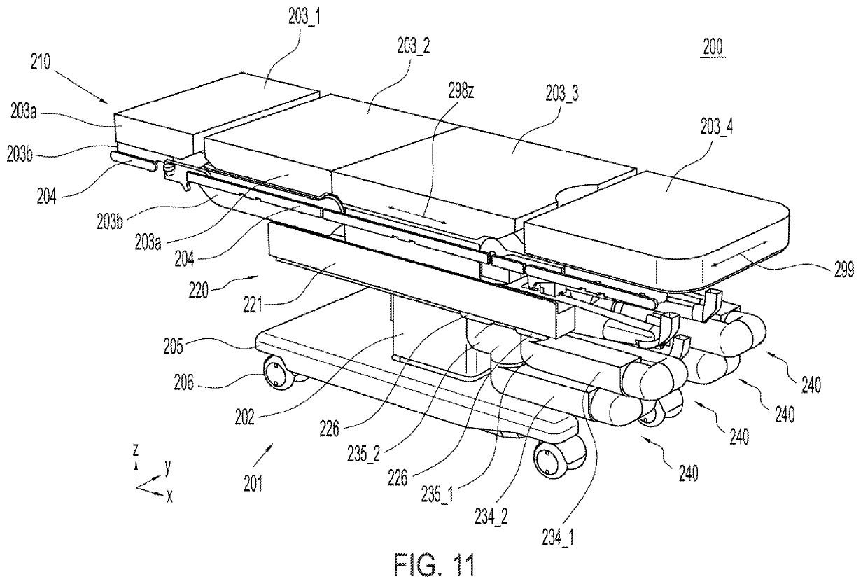

In some embodiments, the motion of the manipulators 140 relative to the platform assembly 110 enabled by the rail assembly 120 allows the distal link assemblies of the manipulators 140 to be moved into a nested configuration near one end portion of the platform assembly 110 which can allow the intermediate and distal link assemblies of the manipulators 140 to be moved around an end of the platform assembly 110 to positions adjacent a laterally extending side 109a of the platform assembly 110. In particular, in some embodiments the nested configuration includes a configuration in which the proximal arms 141 of two adjacent manipulators 140 coupled to the same rail 121 are oriented at angles of 180 degrees or more relative to that rail 121 (the angles measured in the same direction as the angles φ_1 and φ_2 illustrated in FIG. 10, which is described in greater detail below). In other words, the nested configuration includes a configuration in which each of the proximal arms 141 is oriented parallel with, or beyond parallel with, the longitudinal dimension 197 of the rail 121 and/or the longitudinal dimension 199 of the platform 110. Moreover, in the nested configuration, the proximal arms 141 of the two manipulators 140 are positioned adjacent to one another near an end portion of the platform assembly 110 (e.g., near a foot end in some embodiments) with the proximal arms 141 overlapping one another in the vertical direction (e.g., z-axis direction). This allows the manipulators to be out of the way for tasks that need clearance along longitudinally extending sides 109b of the platform assembly 110. In the embodiments of FIG. 1 the manipulators 140 are configured to be moved beyond the foot end of the platform assembly 110 in the nested configuration. In other embodiments, the manipulators 140 can be moved beyond the head end of the platform assembly 110, and in still other embodiments the manipulators 140 can be moved beyond the both the head end and beyond the foot end. In addition, the nested configuration can allow for the manipulators 140 to be stowed in a compact manner under the second end section 103_3.

In some embodiments, the rail assembly 120 can be similar to the rail assemblies described in U.S. Provisional Patent Application No. 63/336,773, entitled “RAIL ASSEMBLY FOR TABLE MOUNTED MANIPULATOR SYSTEM, AND RELATED DEVICES, SYSTEMS AND METHODS,” and in U.S. Provisional Patent Application No. 63/336,778, entitled “NESTING PROXIMAL LINKS FOR TABLE MOUNTED MANIPULATOR SYSTEM, AND RELATED DEVICES, SYSTEMS AND METHODS,” incorporated by reference above.

The user input and feedback system 1004, control system 1006, and auxiliary system 1008 will be further described. Some or all of these components can be provided at a location remote from the table assembly 101. The user input and feedback system 1004 is operably coupled to the control system 1006 and includes one or more input devices to receive input control commands to control operations of the manipulators 140, instruments 150, rails assembly 120, and/or table assembly 101. Such input devices can include but are not limited to, for example, telepresence input devices, triggers, grip input devices, buttons, switches, pedals, joysticks, trackballs, data gloves, trigger-guns, gaze detection devices, voice recognition devices, body motion or presence sensors, touchscreen technology, or any other type of device for registering user input. In some cases, an input device can be provided with the same degrees of freedom as the associated instrument that they control, and as the input device is actuated, the instrument, through drive inputs from the manipulator assembly, is controlled to follow or mimic the movement of the input device, which can provide the user a sense of directly controlling the instrument. Telepresence input devices can provide the operator with telepresence, meaning the perception that the input devices are integral with the instrument. The user input and feedback system 1004 can also include feedback devices, such as a display device (not shown) to display images (e.g., images of the workspace as captured by one of the instruments 1010), haptic feedback devices, audio feedback devices, other graphical user interface forms of feedback, etc.

The control system 1006 can control operations of the system 100. In particular, the control system 1006 can send control signals (e.g., electrical signals) to the table assembly 101, rail assembly 120, manipulators 140, and/or instruments 150 to control movements and/or other operations of the various parts. In some embodiments, the control system 1006 can also control some or all operations of the user input and feedback system 1004, the auxiliary system 1008, or other parts of the system 100. The control system 1006 can include an electronic controller to control and/or assist a user in controlling operations of the manipulator assembly 1001. The electronic controller includes processing circuitry configured with logic for performing the various operations. The logic of the processing circuitry can include dedicated hardware to perform various operations, software (machine readable and/or processor executable instructions) to perform various operations, or any combination thereof. In examples in which the logic includes software, the processing circuitry can include a processor to execute the software instructions and a memory device that stores the software. The processor can include one or more processing devices capable of executing machine readable instructions, such as, for example, a processor, a processor core, a central processing unit (CPU), a controller, a microcontroller, a system-on-chip (SoC), a digital signal processor (DSP), a graphics processing unit (GPU), etc. In cases in which the processing circuitry includes dedicated hardware, in addition to or in lieu of the processor, the dedicated hardware can include any electronic device that is configured to perform specific operations, such as an Application Specific Integrated Circuit (ASIC), a Field Programmable Gate Array (FPGA), a Complex Programmable Logic Device (CPLD), discrete logic circuits, a hardware accelerator, a hardware encoder, etc. The processing circuitry can also include any combination of dedicated hardware and processor plus software.

Differing degrees of user control versus autonomous control can be utilized in the system 100, and embodiments disclosed herein can encompass fully user-controlled systems, fully autonomously-controlled systems, and systems having any combination of user and autonomous control. For operations that are user-controlled, the control system 1006 generates control signals in response to receiving a corresponding user input command via the user input and feedback system 1004. For operations that are autonomously controlled, the control system 1006 can execute pre-programmed logic (e.g., a software program) and can determine and send control commands based on the programming (e.g., in response to a detected state or stimulus specified in the programming). In some systems, some operations can be user controlled and others autonomously controlled. Moreover, some operations can be partially user controlled and partially autonomously controlled—for example, a user input command can initiate performance of a sequence of events, and then the control system 1006 can perform various operations associated with that sequence without needing further user input.

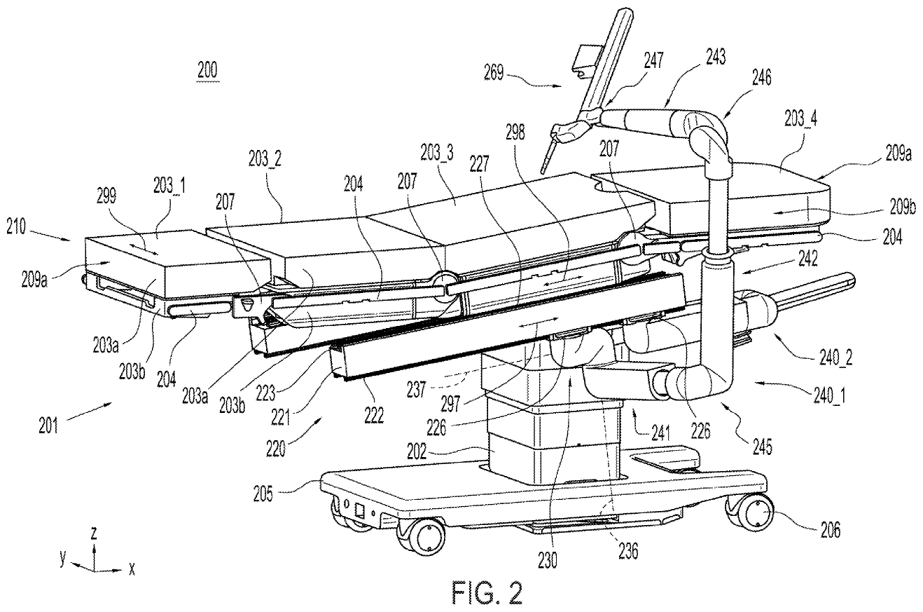

Turning now to FIGS. 2-11, another embodiment of a table-mounted manipulator system 200 (“system 200”) is described below. The system 200 can be used as the system 100, and some components of the system 200 can be used as components of the system 100 described above. In particular, the system 200 includes manipulators 240 (e.g., manipulators 240_1, 240_2) that can be used as the manipulators 140 of the system 100, as described in greater detail below. Thus, the descriptions of the components of the system 100 above are applicable to the related components of the system 200, and duplicative descriptions of these components are omitted below. The related components of the systems 100 and 200 are given reference numbers having the same right-most two digits—for example, 140 and 240. Although the system 200 is one embodiment of the system 100, the system 100 is not limited to the system 200.

As shown in FIG. 2, the system 200 includes a table assembly 201, two rail assemblies 220 coupled to the table assembly, and multiple manipulators 240 coupled to the rail assemblies 220. Each manipulator 240 is configured to support one or more instruments (not illustrated), which can be removably or permanently mounted thereon. The system 200 also can include a control system (not illustrated), a user input and feedback system (not illustrated), and/or an auxiliary system (not illustrated) similar to those described above in relation to the system 100. In some embodiments, the system 200 is configured as a computer-assisted, teleoperable medical system. In other embodiments, the system 200 is configured as a teleoperable system for use in non-medical contexts.

As shown in FIG. 2, the table assembly 201 includes a platform assembly 210 configured to support the patient or inanimate workpiece, a support column 202 coupled to and supporting the platform assembly 210, and a base 205 coupled to the support column 202. The base 205 can be configured to contact the ground or other surface upon which the table assembly 201 rests, and in some embodiments the base 205 includes wheels 206 to allow movement of the system 200 along the ground or other surface. In some embodiments the support column 202 includes a telescoping support column that can raise or lower the platform assembly 210.

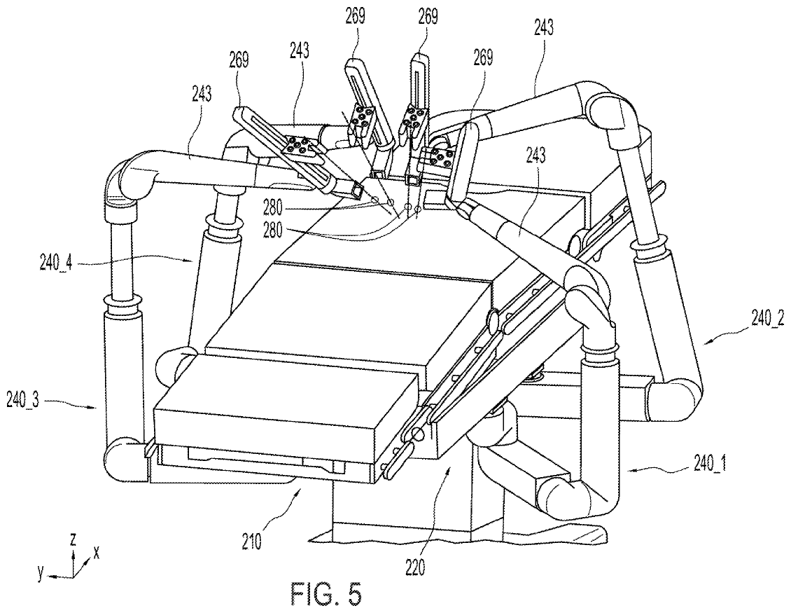

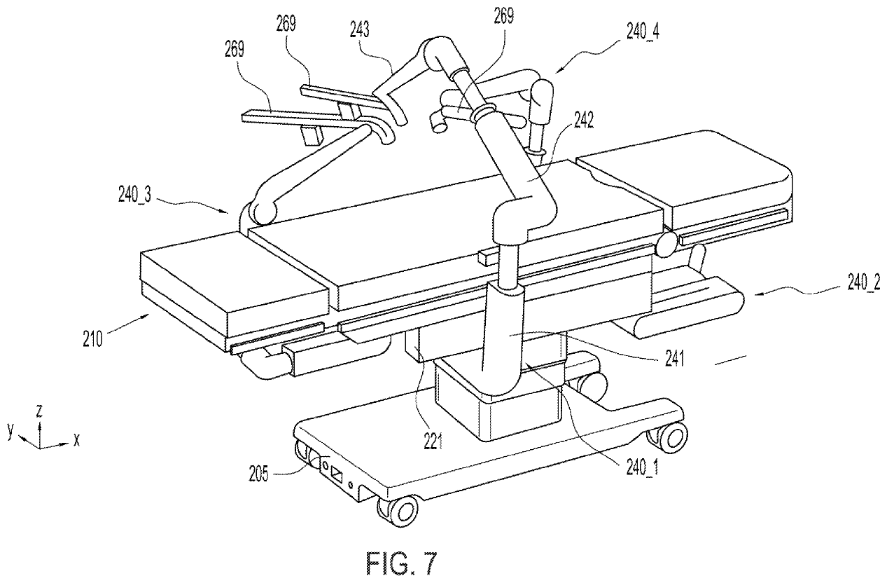

The platform assembly 210 includes multiple platform sections 203 configured to support the patient or workpiece. In particular, in the embodiment illustrated in FIGS. 2-11, the platform assembly 210 includes first end section 203_1 (“head section 203_1”), middle sections 203_2 and 203_3, and second end section 203_4 (“foot section 203_4”), which are arranged in series and movably coupled together via joints 207. In some embodiments, the first end section 203_1 can be configured to support a head of the patient, the second end section 203_4 can be configured to support the feet and/or legs of the patient, and the more middle sections 203_2 and 203_3 can be configured to support a torso and/or other portions of the patient. The joints 207 allow adjacent platform sections 203 to pivot relative to one another about rotation axes parallel to a lateral dimension 299 of the platform assembly 210 (e.g., parallel to a y-axis in the Figures). In some embodiments, one or more of the platform sections 203 can be removable, for example when it is not needed based on the size of the patient or based on the procedure to be performed. In some embodiments, additional platform sections 203 can be added to the platform assembly 210. In some embodiments, auxiliary devices can be coupled to the one or more of the platform sections 203, in addition to or in lieu of one or more of the platform sections 203; for example, leg stirrups can be coupled the middle section 203_3 in lieu of the second end section 203_4 in some embodiments. FIGS. 7, 10, and 11 illustrate the platform assembly 210 in a neutral configuration in which all of the platform sections 203 are parallel to one another and to the ground or other supporting surface upon which the table assembly 201 rests, and FIGS. 2 and 5 illustrates the platform assembly 210 in articulated configurations in which some of the platform sections 203 are oriented at non-zero angles relative to adjacent platform sections 203 and/or relative to the ground or other supporting surface. In some embodiments, some of the joints 207 can also allow for other motion between adjacent platform sections 203, such as relative translation along the longitudinal dimension 298 or relative rotation around a vertical axis parallel to a height dimension (i.e., the z-axis in the Figures), which is perpendicular to the lateral and longitudinal dimensions 299 and 298. In some embodiments, the platform sections 203 include relatively rigid support portions 203b and softer cushion portions 203a attached to the support portions 203b, with a surface of the cushion portions 203a (i.e., the top surface in the orientation illustrated in FIG. 2) forming a support surface that contacts the patient or workpiece. In some embodiments, multiple platform sections 203 can share some components. For example, as illustrated in FIG. 2, the middle platform sections 203_2 and 203_3 can share the same cushion portion 203a that extends across both platform sections 203_2 and 203_3. The cushion portion 203a shared by the platform sections 203_2 and 203_3 can bend when the platform sections 203_2 and 203_3 are articulated relative to one another, as shown in FIG. 2.

In addition to moving individual platform section 203 relative to adjoining platform sections 203, the platform assembly 210 as a whole is movable relative to the support column 202. In some embodiments, the middle section 203_3 is coupled to the support column 202 by one or more joints (not illustrated), providing for motion between the middle section 203_3 and the column 202. The other platform sections 203_1, 203_2, and 203_4 are coupled (directly or indirectly) to the middle section 203_3, and thus as the middle section 203_3 moves relative to the support column 202 the platform assembly 210 as a whole moves relative to the support column 202. In some embodiments, the motion of the middle section 203_3 (and hence platform assembly 210 as a whole) relative to the support column 202 includes pivoting (tilting) about a horizontal axis parallel to the lateral dimension 299 (e.g., a pitch degree of freedom of motion), as shown in FIG. 2. In some embodiments, other degrees of freedom of motion are provided between the middle section 203_3 and the support column 202, including pivoting (tilting) about a horizontal axis parallel to the longitudinal dimension 298 (e.g., a roll degree of freedom of motion), rotating about a vertical axis (e.g., a yaw degree of freedom of motion), and/or translation along the longitudinal and/or lateral dimensions 299 or 298. As shown in FIGS. 7, 10, and 11, in the neutral configuration of the platform assembly 210, the platform assembly 210 is parallel to the ground or other supporting surface.

As shown in FIG. 2, the platform assembly 210 also includes a number of accessory rails 204 attached to side surfaces of the support portions 203b of the platform sections 203. The accessory rails 204 can be configured to receive accessory devices removably mounted thereon, such as such as leg stirrups, liver retractor, arm boards, and bed extenders. The accessory rails 204 are be attached to longitudinally extending side faces of one or more of the platform sections 203.

As noted above, the system 200 includes multiple manipulators 240. In the embodiment illustrated in FIGS. 2-11, four manipulators 240 are present, with two manipulators 240 on each longitudinally extending side 209b of the platform assembly 210 (i.e., two manipulators 240 are mounted to a first longitudinally extending side 209b of the platform assembly 210 and two manipulators 240 are mounted to a second longitudinally extending side 209b of the platform assembly 210). In other embodiments, more or fewer manipulators 240 can be used, such as one, two, three, or more manipulators per longitudinally extending side 209b. In FIG. 11, the manipulators 240 are shown in a stowed state under end section 203_4 while remaining coupled to the rail 221, while in FIGS. 2-10 some or all of the manipulators 240 are shown in various deployed states (not all of the manipulators 240 are visible in each of the Figures). The deployed states include states in which one or more of the manipulators 240 are not stowed, which in some embodiments means at least that the one or more manipulators 240 are at least partially unfolded/uncompacted while remaining coupled to the rail 221, and removed from a stowed location, e.g., removed out from under the platform assembly 110. The manipulators 240 can be positioned in a variety of deployed states. In some deployed states, the manipulators 240 are deployed and have a distal link assembly positioned in a sterile field (see, for example, FIG. 2). The sterile field is a region in which any exposed surfaces of objects in the region are maintained in a sterile condition (e.g., a condition substantially free from contaminants, such as biological pathogens, dusts, oils, etc.) and non-sterile surfaces are covered by a sterile barrier. The sterile field usually includes regions that are above a certain height, such as above the rail 221, platform 210, or other chosen reference point, while portions below that height are considered to not be in the sterile field. In some deployed states the distal link assembly 263 is raised to a height sufficient for the distal link assembly 263 and any instrument supported thereon to remain within the sterile field while deployed. Thus, in some embodiments in the deployed state, the distal link assembly 263 is at or above a height of the proximal arm 241, the rail 221, the platform 210, and/or some other predetermined level. In other deployed states, the manipulators 240 can be deployed, but do not have a distal link assembly positioned fully in the sterile field (see, for example, FIG. 10). The deployed state can include a variety of configurations and positions of the manipulators 240 including but not limited to those shown in FIGS. 2-10.

The manipulators 240 include a number of links moveably coupled together via joints, as described above in relation to the manipulators 140. In particular, FIGS. 3 and 4 illustrate two embodiments of manipulators 240_1 and 240_2, respectively. The manipulators 240_1 and 240_2 are generally similar to one another except that the manipulator 240_1 includes two proximal joints 230 and associated joint housings 264 and 265, and a proximal arm 241 of the manipulator 240_1 is positioned at a lower height than the proximal arm 241 of the manipulator 240_2, as described in greater detail below. In some embodiments, one manipulator 240 of each rail 221 is configured like the manipulator 240_1 illustrated in FIG. 3 while the other manipulator 240 of each rail is configured like the manipulator 240_2 illustrated in FIG. 4. In other embodiments, multiple manipulators 240 coupled to the same rail 221 have two proximal joints 230 similar to the manipulators 240_1, in particular in some embodiments all of the manipulators 240 have two proximal joints 230 similar to the manipulators 240_1. In still other embodiments, none of the manipulators 240 coupled to a given rail 221 (including, in some embodiments, none of the manipulators 240 in the entire system 200), have two proximal joints 230; for example, in some of these embodiments all of the manipulators 240 are configured similarly to the manipulator 240_2. Descriptions below of various parts of the manipulators 240 below are applicable to both types of manipulators 240_1 and 240_2 unless otherwise noted as applying specifically to one type or the other.

As shown in FIGS. 3 and 4, each manipulator 240 includes a proximal link assembly 261 including a proximal arm 241 coupled to the rail assembly 220 via one or more proximal joints 230 and a carriage 226, an intermediate link assembly 262 including an intermediate arm 242 coupled to a distal end portion of the proximal link assembly 261 via one or more intermediate joints 245, and a distal link assembly 263 including a distal arm 243 coupled to the intermediate link assembly 262 via one or more distal joints 246. The distal link assembly 263 also includes an instrument holding portion 269 coupled to the distal arm 243 and configured to support the instrument 250.

As noted above, the proximal link assembly 261 includes a proximal arm 241. As shown in FIGS. 3 and 4, the proximal arm 241 includes a first link 241a and a second link 241b. The first and second links 241a and 241b are translatable relative to one another along a direction 248 parallel to a longitudinal dimension of the proximal arm 241. In the illustrated embodiment, the first link 241a and second link 241b are in a telescoping arrangement to provide the translational degree of freedom movement, with the second link 241b received within a bore of the first link 241a, but in other embodiments the opposite arrangement is used with the first link 241a received in a bore of the second link 241b. In still other embodiments the first and second links 241a and 241b are arranged in configurations in which the links 241a and 241b are translatable relative to one another but one is not received within the other, such as a side-by-side configuration in which the links 241a and 241b are positioned adjacent to one another or a one-above-the-other configuration in which the links 241a and 241b are aligned together in series (e.g., end to end).

As shown in FIGS. 3 and 4, a proximal end portion of the proximal arm 241 of each manipulator 240 is coupled to a carriage 226 (also “first carriage 226”) via a first proximal joint 230a. The carriage 226 is in turn coupled to the rail 221, as shown in FIG. 2. The first proximal joint 230a allows for rotation of the proximal arm 241 relative to the carriage 226 (and hence relative to the rail 221) about a first axis 236 that is perpendicular a longitudinal dimension 297 of the rail 221, as shown in FIGS. 2-4. The first axis 236 is also perpendicular to both a longitudinal dimension 297 and a lateral dimension 299 of the platform 210, or in other words is oriented vertically, in a neutral position of the table assembly 201 and in a neutral state of the proximal arm 241.

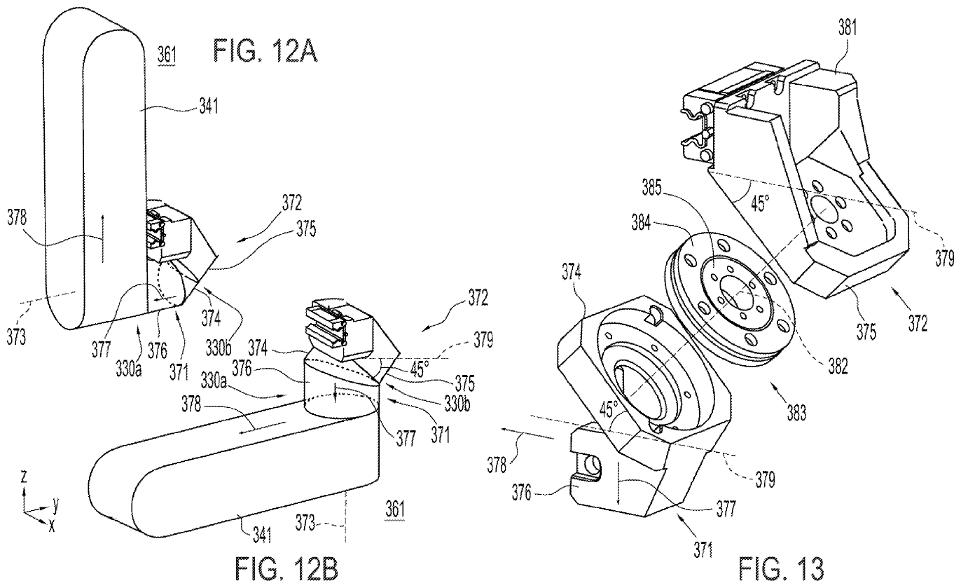

As noted above, one difference between the manipulator 240_1 of FIG. 3 and the manipulator 240_2 of FIG. 4 is that the manipulators 240_1 further include a second proximal joint 230b interposed between the first proximal joint 230a and the carriage 226, whereas in the manipulator 240_2 the first proximal joint 230a is coupled directly to the carriage 226 without a second proximal joint 230b. The second proximal joint 230b provides for rotation of the proximal arm 241 relative to the rail 221 around a second axis 237 orthogonal to the first axis 236 and parallel to a longitudinal dimension 297 of the rail 221 (i.e., the second axis 237 is horizontal when the table assembly 101 is in a neutral position). More specifically, a first proximal joint housing 264 is rotatably coupled to a second proximal joint housing 265 via the second proximal joint 230b, allowing the second proximal joint housing 265 to rotate relative to the first proximal joint housing 264 around the second axis 237. The first proximal joint housing 264 is coupled to the carriage 226, and the second proximal joint housing 265 is rotatably coupled to the proximal arm 241 via the first proximal joint 230a. In the embodiment illustrated in FIGS. 2-11, the first and second proximal joint housings 264 and 265 each form L-shaped 90-degree bends. In other embodiments, different joint arrangements can be used for the proximal joints 230, such as in the embodiment of FIG. 12A-12B described in greater detail below in which a 45-degree rotary joint is used as the second proximal joint. As another example, in some embodiments one or more manipulators 240 can be provided with one or more proximal joints 230 allowing for rotation of the proximal arm 241 about the axes 236 and 237, like the manipulator 240_1, but unlike the manipulator 240_1 the proximal joint(s) 230 in these manipulators 240 are configured such that the second axis 237 is not significantly offset from a central longitudinal axis of the proximal arm 241 (e.g., the second axis 237 and central longitudinal axis of the proximal arm 241 interest and are within the same plane). Moreover, in still other embodiments none of the manipulators 240 coupled to a same rail 221 (or none of the manipulators 240 in the entire system 200, in some embodiments) are provided with the ability to rotate about the axis 237, for example, where multiple manipulators 240_2 are present while manipulators 240_1 are not provided.

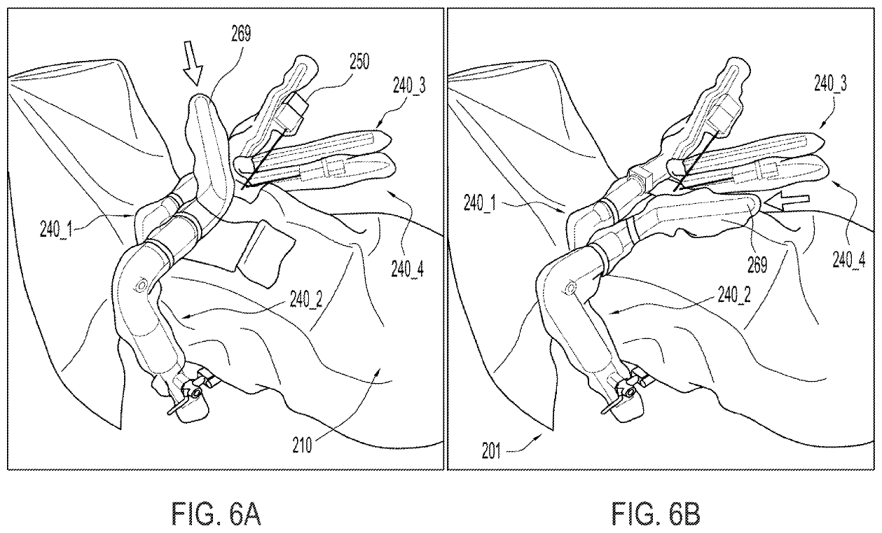

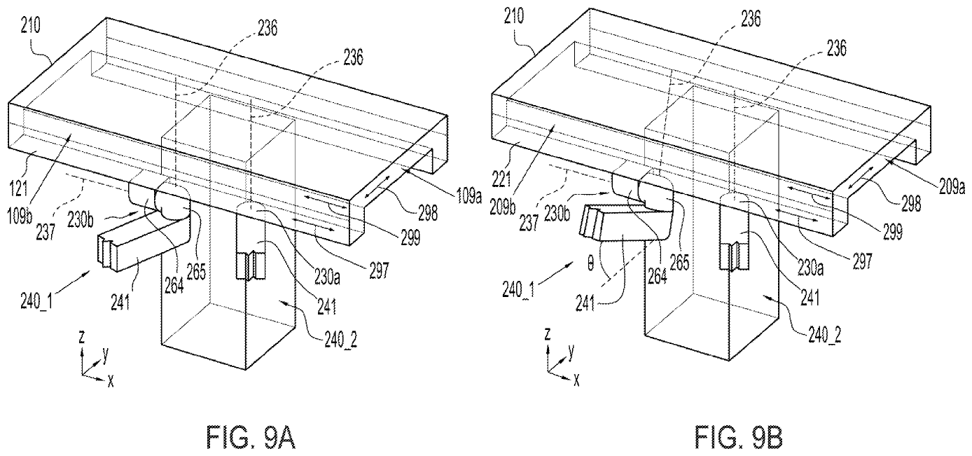

Rotation of the proximal arm 241 around the second axis 237 (e.g., via second proximal joint 230b) causes the proximal arm 241 to incline or decline relative to a horizontal plane (e.g., a plane parallel to the longitudinal or lateral dimensions 298 and 299 of the platform 210 in embodiments in which the rail assembly 220 is coupled to the platform 210, or a plane parallel to the ground in embodiments in which the rail assembly 220 is coupled to the support column 202), thus raising or lowering a distal end of the proximal arm 241 relative to the rail 221. For example, FIG. 9A illustrates the proximal arm 241 in a neutral position in which the proximal arm 241 is parallel to a horizontal plane defined by the longitudinal and lateral dimensions 298 and 299, and FIG. 9B illustrates the proximal arm 241 in an inclined position in which the proximal arm 241 has been rotated about the second axis 237 (e.g., via second proximal joint 230b) and is inclined at a positive angle θ relative to the horizontal plane. As the proximal arm 241 inclines relative to the horizontal plane, movement of the proximal arm 241 can cause more distal portions of the manipulator to correspondingly both raise and extend further across the table (as opposed to vertical movement alone). The ability to rotate about the second axis 237 (e.g., via second proximal joint 230b) allows the proximal arm 241 to be moved between orientations ranging at least between a horizontal orientation and a vertical inclined orientation (e.g., at least 90 degrees of rotation or any angles in between). For example, FIGS. 6 and 7 illustrate a state in which the proximal arm 241 of one manipulator 240 (labeled 240_1 in FIGS. 6 and 7) is inclined to a fully vertical orientation (i.e., parallel to a height dimension of the platform 210, or the z-axis in the Figures). In some embodiments, the ability to rotate about the second axis 237 (e.g., via the second proximal joint 230b) can also allow for rotation of the proximal arm 241 to orientations that are declined relative to a horizonal orientation.

Another difference between the manipulators 240_1 and 240_2 is that the proximal arm 241 of the manipulator 240_1 is positioned at a different height than the proximal arm 241 of the manipulator 240_2 relative to the rail 221, as best seen in FIGS. 10 and 11. Specifically, the proximal arm 241 of the manipulator 240_1 is positioned further in a vertical (z-axis) direction away from the rail 221 (i.e., closer to the ground or other supporting surface) than the proximal arm 241 of the manipulator 240_2. More specifically, the difference in height between these proximal arms 241 is sufficiently large that the lower proximal arm 241 of the manipulator 240_1 can be moved under the higher proximal arm 241 of the manipulator 240_2 without collision, as illustrated in FIGS. 10 and 11. This difference in height is determined by the height dimensions of the first and second proximal joint housings 264 and 265. The height difference allows the manipulators 240 to be placed in a nested configuration, as in FIGS. 10 and 11. In particular, the nested configuration includes a configuration in which the proximal arms 241 of two adjacent manipulators 240_1 and 240_2 coupled to the same rail 221 are oriented at angles φ_1 and φ_2 of 180 degrees or more relative to that rail 221, as shown in FIG. 10 (the angles referred to herein are measured in the directions illustrated in the FIG. 7). In other words, the nested configuration includes a configuration in which each of the proximal arms 241 is oriented parallel with, or beyond parallel with, the longitudinal dimension 297 of the rail 221 and/or the longitudinal dimension 298 of the platform 210, as shown in FIGS. 10 and 11. Moreover, in the nested configuration, the proximal arms 241 overlap one another in the vertical direction (e.g., z-axis direction). In some circumstances, the nested configuration allows the more distal portions of the manipulators 240 in a deployed state to be swung around the end of the platform 210 such that the manipulators 240 are moved away from the longitudinally extending side 209b of the platform assembly 210 to locations along the laterally extending side 209a of the platform, as shown in FIG. 7. The nested configuration with the manipulators 240 deployed and positioned along the laterally extending side 209a can be beneficial for various tasks that require or benefit from free space along the longitudinally extending sides 109b, such a transferring a patient from a patient transport gurney to the platform assembly 210. The nested configuration also allows for the manipulators 240 to be stowed in a compact manner under the second end section 203_4, as shown in FIG. 11. It should be understood that, in some embodiments, offsets between the heights of adjacent proximal arms 241 such as those described above could be provided using different numbers, shapes, and/or arrangements of proximal joints and joint housings than those depicted in FIGS. 2-4. Moreover, although in the embodiment depicted in FIGS. 2-4 the proximal arm 241 of the manipulators 240_1 is vertically offset at least in part due to the inclusion of the second proximal joint 230b, in other embodiments manipulators 240 that lack such a second proximal joint (or which lack the ability to rotate about the second axis 237) can nevertheless have their proximal arm 241 vertically offset, for example by providing a proximal joint housing that extends vertically down from the carriage 226. Furthermore, in some embodiments, no vertical offset is provided between the proximal arms 241 of adjacent manipulators 240.

Returning to FIGS. 2-4, the intermediate link assembly 262 includes an intermediate arm 242. As shown in FIGS. 3 and 4, the intermediate arm 242 includes a first link 242a and a second link 242b. The first and second links 242a and 242b are translatable relative to one another along a direction 249 parallel to a longitudinal dimension of the intermediate arm 242. In other words, the first and second links 242a and 242b are coupled together by a prismatic joint. In the illustrated embodiment, the first link 242a and second link 242b are in a telescoping arrangement to provide the translational degree of freedom movement, with the second link 241b received within a bore of the first link 242a, but in other embodiments the opposite arrangement is used with the first link 242a received in a bore of the second link 242b, and in still other embodiments the first and second links 242a and 242b are arranged side-by-side instead of one being received within the other.

As shown in FIGS. 3 and 4, a proximal end portion of the intermediate arm 242 of each manipulator 240 is rotatably coupled to the second link 241b of the proximal arm 241 via a first intermediate joint 245a. More specifically, a proximal end portion of the intermediate arm 242 is rotatably coupled to an intermediate joint housing 266 via the first intermediate joint 245a, with the intermediate joint housing 266 being coupled to or a part of a distal end portion of the proximal arm 241. The first intermediate joint 245a allows for rotation of the intermediate arm 242 relative to the proximal arm 241 about a third axis 238, which is perpendicular to the longitudinal dimension of the proximal arm 241 and the longitudinal dimension of the intermediate arm 242. In addition, a second intermediate joint 245b is provided to allow for rotation of a distal portion of the intermediate arm 242 relative to a proximal portion of the intermediate arm 242 about a fourth axis 239 that is parallel to the longitudinal dimension of the intermediate arm 242. For example, in the embodiment illustrated in FIGS. 3 and 4, the second intermediate joint 245b rotatably couples the first link 242a to the second link 242b such that the second link 242b can rotate relative to the first link 242a about the fourth axis 239 while also allowing for translation between the first link 242a and 242b. In other words, in the embodiment of FIGS. 3 and 4, the second intermediate joint 245b serves as both the prismatic joint between the first and second links 242a and 242b that was mentioned above and also as a rotary joint between the first and second links 242a and 242b. In other embodiments, the second intermediate joint 245b can serve solely as a rotational joint and/or can be provided at a different location in the intermediate link assembly 262 instead of being provided between the first and second links 242a and 242b. For example, in some embodiments (not illustrated), the first link 242a is split into two parts: a proximal part rotatably coupled to the proximal arm 241 via first intermediate joint 245a and a distal part rotatably coupled to the proximal part via the second intermediate joint 245b. The distal part of the first link 242a can then movably coupled to the second link 242b via a prismatic joint as described above. Thus, in such embodiments, the prismatic joint between the first link 242a and the second link 242b is disposed distally of the second intermediate joint 245b. In still other embodiments, the second link 242b is split into two parts which are rotatably coupled together by the second intermediate joint 245b, in which case the prismatic joint between the first and second links 242a and 242b is disposed proximally of the second intermediate joint 245b.

The distal link assembly 263 includes a distal arm 243, a wrist 247, and an instrument holding portion 269 coupled to the distal arm 243 via the wrist 247. As shown in FIGS. 3 and 4, a proximal end portion of the distal arm 243 of each manipulator 240 is rotatably coupled to the second link 242b of the intermediate arm 242 via a first distal joint 246a. More specifically, a distal end of the second link 242b is coupled to or includes a first distal joint housing 267, which is rotatably coupled to a second distal joint housing 268 that is coupled to or part of the distal arm 243. The first distal joint 246a allows for rotation of the distal arm relative to the intermediate arm 242 about a fifth axis 252, which is perpendicular to the longitudinal dimension of the intermediate arm 242 and the longitudinal dimension of the distal arm 243. In addition, a second distal joint 246b rotatably couples the wrist 247 to the second distal joint housing 268 (via the arm 243) such that the wrist 247 can rotate relative to the second distal joint housing 268 about a sixth axis 251, which is parallel to the longitudinal dimension of the distal arm 243. Rotation about this sixth axis 251 via the second distal joint 246b constitutes a degree of freedom of motion of the wrist 247, which can be referred to as roll. Thus, the sixth axis 251 can also be called a roll axis. In some embodiments, the distal arm 243 moves along with the wrist 247 as the wrist rotates around the sixth axis 251 (i.e., the distal arm 243 rotates relative to the second distal joint housing 268), and in other embodiments the distal arm 243 remains stationary relative to the second distal joint housing 268 as the wrist rotates around the sixth axis 251 (i.e., the wrist 247 rotates relative to the distal arm 243).

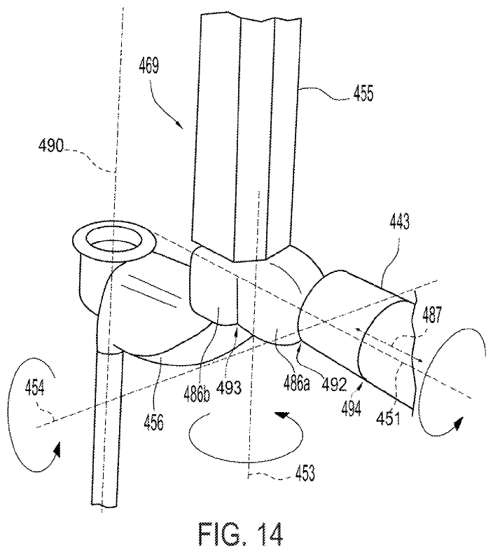

In addition to the roll degree of freedom of motion described above, the wrist 247 allows for rotation of the instrument holding portion 269 relative to the arm 243 about two additional axes, the seventh and eighth axes 253 and 254. The seventh and eight axes 253 and 254 are perpendicular to one another. The seventh and eight axes 253 and 254 are also perpendicular to the sixth axis 251 and hence also to the longitudinal dimension of the distal arm 243 in a neutral state of the wrist 247, but not necessarily in other states of the wrist 247. Specifically, in some embodiments, the seventh axis 253 remains perpendicular to the sixth axis 251 in all states of the wrist, whereas the eight axis 254 does not. In other embodiments, this relationship is reversed, with the eight axis 254 remaining perpendicular to the sixth axis 251 while the seventh axis 253 does not. Rotation about the seventh and eighth axes 253 and 254 can be referred to as pitch and yaw degrees of freedom of motion, respectively, and thus the seventh and eighth axes 253 and 254 can be referred to as pitch and yaw axes, respectively. In particular, the wrist 247 includes two wrist joints that provide for rotation about the seventh and eighth axes. FIG. 14, described in greater detail below, illustrates an embodiment of a wrist mechanism that can be used as the wrist 247.

Some of the degrees of freedom of motion of the manipulators 240 described above are redundant in the sense of not being strictly necessary in order to manipulate the instrument, but inclusion of these redundant degrees of freedom of motion can improve manipulation of the manipulators 240 in some circumstances, for example by helping with reach and collision avoidance. For example, the rotation of the wrist 247 about the eighth axis 254 (yaw), the extension of the proximal arm 241 along direction 248, and the extension of the intermediate arm 242 along the direction 249 can be redundant degrees of freedom of motion. In some embodiments, one, some, or all of these degrees of freedom of motion are omitted. In some embodiments, some or all of these redundant degrees of freedom of motion can be used as setup joints whose position is fixed so as to obtain a desired deployed pose of the manipulator 240 but which thereafter remain stationary during the procedure (or are only moved infrequently during the procedure when a change of pose is desired).

As shown in FIGS. 3 and 4, the instrument holding portion 269 includes a instrument holder base member 255 coupled to the wrist 247 and extending parallel to the eighth axis 254, an instrument holder 244 movably coupled to the instrument holder base member 255, and an accessory mount portion 256 coupled to one end portion of the instrument holder base member 255. The instrument holder 244 is translatable along a length of the instrument holder base member 255 along a direction parallel to the eighth axis 254. The instrument holder 244 includes an interface to couple to an instrument 250 mounted thereto. For example, the interface can include output couplers (not illustrated) to engage (directly or indirectly via an intermediary) with input couplers (not illustrated) of the instrument 250 to provide driving forces or other inputs to the mounted instrument 250 to control various degree of freedom movement and/or other functionality of the instrument 250. The accessory mount portion 256 is configured to receive an accessory mounted thereon, such as a cannula. The cannula mounted to the accessory mount portion 256 can be positioned to receive an instrument shaft of an instrument 250 mounted to the instrument holder 244. The instrument shaft and a passage through the cannula can define an insertion axis along with the instrument can translate in response to translation of the instrument holder 244 along the instrument holder base member 255. A remote center of motion can be located on the insertion axis in, at, or near the cannula.

The configuration of the manipulator 240 described above provides a wide range of motion of the manipulators and allows the manipulators 240 to be positioned and posed in a variety of poses, which can allow an instrument carried by the manipulators 240 to reach positions (e.g., entry ports) that might otherwise not be possible. In particular, at least the aspects noted above related to the ability of the proximal arm 241 to rotate about the second proximal joint 230b, the ability of the proximal arm 241 to extend, the ability of the intermediate arm 242 to extend, and/or the degrees of freedom of motion provided by the wrist 247, individually or in any combination, can facilitate the positioning and posing of the manipulators 240. For example, FIGS. 5-6B illustrate states in which all four manipulators 240 are deployed and arranged so as to position the shafts of instruments 250 (not illustrated in FIG. 5) carried thereon to utilize four entry ports 280 disposed in a line extending parallel to a lateral dimension 299 of the platform near a middle portion of the platform 210 (e.g., along a patient's abdomen) (hypothetical locations of entry ports 280 are illustrated in FIGS. 5-6B, but actual locations could vary from case to case as desired). As shown in FIG. 5, in order for the shafts of the instruments 250 to reach the entry ports 280, the respective instrument holding portions 269 of the manipulators 240 can need to be arranged in a variety of different poses relative to their respective distal arms 243, and such positioning and posing of the instrument holding portions 269 can be facilitated by the degrees of freedom of motion provided by the wrist 247 described above. Without the wrist 247, such positioning and posing of the instrument holding portions 269 can be difficult, or in some circumstances not possible. For example, the distal arms 243 and instrument holding portions 269 of the manipulators 240_1 and 240_3 can block the manipulators 240_2 and 240_4 and prevent the manipulators 240 from being positioned in a line, but the degrees of freedom of motion provided by the wrist 247 allows the instrument holding portions 269 of the manipulators 240_2 and 240_4 to be oriented as shown in FIG. 5 so that the shafts of the instruments 250 of all of the manipulators 240 can reach the line of entry ports 280 despite the instrument holding portions 269 not being arranged in line. Although the above description focuses on an example in which just one manipulator 240 from each rail 221 is able to reach over the platform 210 to the opposite side, in some embodiments multiple manipulators coupled to the same rail (including all manipulators, in some embodiments) can be configured to be able to reach over to the opposite side of the platform 210. For example, in some embodiments, four manipulators configured similarly to the manipulator 240_1 are provided, with two coupled to each longitudinally extending side of the platform via two rail assemblies, and all four of these manipulators can be able to reach over the platform and patient to position instruments mounted thereon so as to reach entry ports on an opposite side of the patient.

Furthermore, the wrist 247 can also allow for increased range of movement of a distal end of the instrument 250 within the patient. For example, FIGS. 6A and 6B illustrate the manipulator 240_2 moving between two states while an instrument shaft of the instrument 250 is inside the patient. In FIG. 6A, the instrument holding portion 269 of the manipulator 240_2 is positioned somewhat upright, allowing a distal end of the shaft of the instrument 250 to reach locations inside of the patient near the entry port 280. In FIG. 6B, the instrument holding portion 269 of the manipulator 240_2 has been moved from the position in FIG. 6A to a relatively declined orientation, allowing a distal end of the shaft of the instrument 250 to reach locations inside of the patient that are further from the entry port 280. Such movement of the instrument holding portion 269 between these two states is facilitated by the degrees of freedom of motion of the wrist 247. Moreover, in some embodiments, once set in a desired pose, the proximal and intermediate arms 241 and 242 (and in some embodiments the distal arm 243 also) do not move at all (or in some embodiments move very little) during performance of subsequent tasks of the overall procedure, and thus after setting the initial pose subsequent positional changes of the instrument 250 occur exclusively (or in some embodiments primarily) through motion of the wrist 247. This can allow personnel and/or equipment to be positioned near the more proximal and intermediate portions of the manipulators 240 during the procedure without concern of the manipulators 240 subsequently moving and colliding with the personnel or equipment, because it can be known in advance that only the instrument holding portion 269 of the manipulator 240 will be moving around during the procedure.

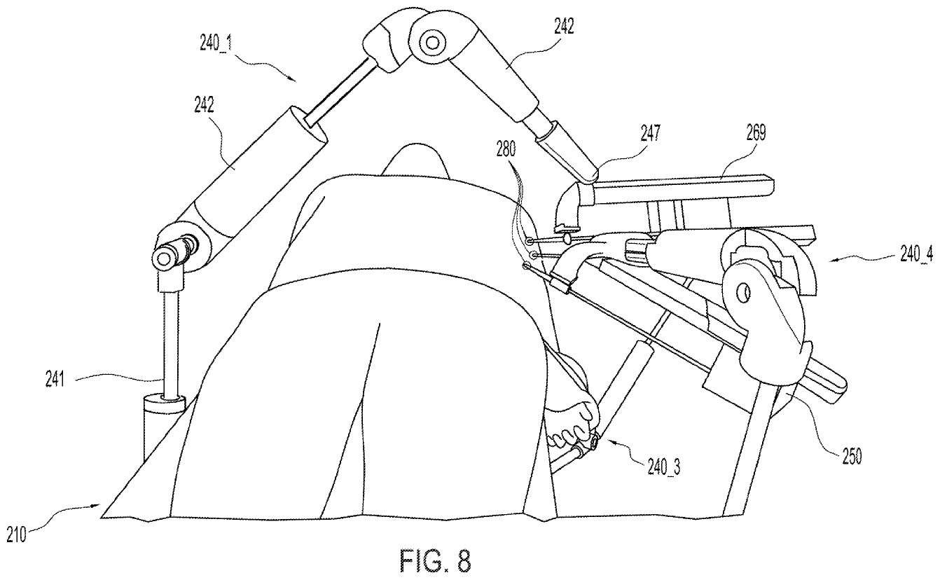

As another example of how the configuration of the manipulator 240 described above provides a wide range of motion of the manipulators 240 and allows the manipulators 240 to be posed in a variety of poses, FIGS. 7 and 8 illustrate a state in which one of the manipulators 240_1 reaches over and across the platform 210 and the patient supported thereon such that the instrument holding portion 269 is at position on an opposite side of the platform 210 from the one the manipulator 240_1 is attached to. This allows the instrument 250 carried by the manipulator 240_1 to reach an entry port 280 on the opposite side of the patient. Thus, the system 200 can be able to perform a procedure with three entry ports 280 arranged on one side of the patient, as shown in FIG. 8, even with only two manipulators 240 positioned along the same side as the entry ports 280. The reaching of the manipulator 240_1 over the platform 210 is facilitated in part by the second proximal joint 230b. In particular, by rotating the proximal arm 241 about the second axis 237 via the second proximal joint 230b, the proximal arm 241 inclines relative to the horizonal plane as shown in FIG. 9B, which raises the distal end of the proximal arm 241. This raising of the distal end of the proximal arm 241 can position the intermediate arm 241 coupled thereto at a height above the patient, such that the intermediate arm 242 can extend at least partially in a lateral direction from the proximal arm 241 across the platform 210 and the patient, as shown in FIG. 8. Moreover, the ability of the proximal arm 241 to extend (increase in length) can be used in the aforementioned inclined state to raise the distal end of the proximal arm 241 even higher than would be possible from only inclining the proximal arm 241, as shown in FIG. 8, which can further aid in positioning the intermediate arm 242 at a height sufficient to allow the intermediate arm 242 to extend at least partially in a lateral direction above and across the patient (this can be particularly useful when additional vertical clearance is needed, such as with a larger patient). With the intermediate arm 242 positioned above and extending partially across the patient, the distal arm 243 can be oriented to extend from the intermediate arm 242 the rest of the way across the patient so as to position the instrument holding portion 269 along the opposite side of the platform 210, as shown in FIG. 8. Moreover, the ability of the intermediate arm 242 to extend (increase in length) can be used in this configuration to increase the distance across which the intermediate arm 242 and distal arm 243 can reach in the lateral direction, which can further aid in positioning the instrument holding portion 269 along the opposite side of the platform 210 (this can be particularly useful when additional lateral reach is needed).

As shown in FIG. 2, the manipulators 240 are coupled to the table assembly 201 via the rail assemblies 220. One rail assembly 220 is provided for each of the two longitudinally extending sides of the platform assembly 210. The description below describes a single rail assembly 220 for ease of description, and the other rail assembly 220 can be configured similarly. As shown in FIG. 2, the rail assembly 220 includes a movable rail 221 (“rail 221”), first carriages 226 coupled to the rail 221 and to the manipulators 240 (at least one first carriage 226 per manipulator 240), and one or more second carriages 227 coupled to the rail 221 and the table assembly 201. Each first carriage 226 couples a respectively corresponding one of the manipulators 240 to the rail 221 such that the manipulators 240 can translate relative to the rail 221 along a longitudinal dimension 297 of the rail 221. In addition, each second carriage 227 couples the rail assembly 220 to the platform assembly 210 (e.g., to the middle section 203_3) such that the rail 221 can translate relative to the platform assembly 210 and the support column 202 along the longitudinal dimension 297 of the rail 221. Specifically, in the embodiment illustrated in FIGS. 2-11, the one or more second carriages 227 couple the rail 221 to the middle section 203_3, and thus in this embodiment the longitudinal dimension 297 of the rail 221 is parallel to the longitudinal dimension 298 of the platform assembly 210 regardless of how the platform assembly 210 is moved or oriented relative to the support column 202. In other embodiments, the rail assembly 220 can be coupled to another platform section 203. In still other embodiments the rail assembly 220 can be coupled directly to the support column 202, and thus in these embodiments the orientation of the longitudinal dimension 297 of the rail 221 relative to the longitudinal dimension 298 of the platform assembly 110 can vary depending on the orientation of the platform assembly 210 relative to the support column 202.

As shown in FIG. 2, the movable rail 221 includes first and second sets of engagement features 222 and 223 configured to engage with complementary engagement features of the first and second carriages 226 and 227, respectively, to movably couple the rail 221 to the first and second carriages 226 and 227. The first set of engagement features 222 is configured to engage with complementary engagement features of the first carriages 226, while the second set of engagement features 223 is configured to engage with complementary engagement features of the second carriages 227. The first and second sets of engagement features 222 and 223 each includes two tracks including flanges and/or grooves extending along the longitudinal dimension 297, and the complementary engagement features of the first and second carriages 226 and 227 are configured to engage and ride along the flanges and/or in the grooves of the first and second sets of engagement features 222 and 223, respectively. The tracks of the first set of engagement features 222 are spaced apart from one another in the lateral dimension to provide increased stability to and strengthen the coupling with the first carriages 226. The second set of engagement features 223 is configured similarly. In some embodiments, the complementary engagement features of the first and second carriages 226 and 227 can include bearing devices (not illustrated) configured to reduce friction to facilitate easier translation, such as wheels, balls, plain bearing surfaces, etc.

In some embodiments, the rail assembly 220 further includes translation mechanisms configured to drive translation of the first carriages 226 relative to the rail and to drive translation of the rail 221 relative to the second carriage 227. The translation mechanisms can include actuators, such as electrical motors or other actuation devices (e.g., hydraulic, pneumatic, or other devices to provide a motive force).

The ability to translate the manipulators 240 and the rail 221 relative to the table assembly 201 allows the manipulators 240 to be moved to a variety of positions. In particular, in one state the rail 221 and the manipulators 240 can be moved towards one end of the table assembly 201 (e.g., towards a foot end) to facilitate placing the manipulators 240 in the nested configuration described above and illustrated in FIG. 7.

Turning now to FIGS. 12A-13, an embodiment of a proximal link assembly 361 is described below. The proximal link assembly 361 can be used as a proximal portion of one or more of the manipulators 140 of the system 100 described above. Moreover, the proximal link assembly 361 can be used in lieu of the proximal link assembly 261 in the system 200 described above. Thus, the descriptions of similar components above are applicable to the related components of the proximal link assembly 361, and duplicative descriptions of these components are omitted below. The related components are given reference numbers having the same right-most two digits—for example, 141 and 341.

As shown in FIG. 12A, the proximal link assembly 361 includes a proximal arm 341, a first 45-degree joint part 371, a first proximal joint 330a rotatably coupling the proximal arm 341 to the first 45-degree joint part 371, a second 45-degree joint part 372, and a second proximal joint 330b rotatably coupling the first 45-degree joint part 371 to the second 45-degree joint part 372. The second 45-degree joint part 372 can be coupled to, or can include, a carriage 326, which can be used to couple the proximal link assembly 361 to a rail, such as the rails 121 or 221 described above. The first proximal joint 330a allows the proximal arm 341 to rotate relative to the first 45-degree joint part 371 about an axis 373, which is perpendicular to a longitudinal dimension of the proximal arm 341.

The first and second 45-degree joint parts 371 and 372 each include engagement portions 374 and 375, respectively, that are angled at 45 degrees relative to a horizontal plane 379. The horizontal plane 379 is parallel to longitudinal and lateral dimensions of a rail to which the proximal link assembly 361 is coupled (or couplable). The engagement portions 374 and 375 are parallel to another when the first and second 45-degree joint parts 371 and 372 are coupled together, as illustrated in FIGS. 12A-13, and thus a plane of engagement between the engagement portions 374 and 375 is oriented at an angle of 45 degrees relative to the horizontal plane 379, as shown in FIG. 12B. The first 45-degree joint part 371 further includes an arm coupling portion 376 coupled to the proximal arm 341 and coupled to the engagement portion 374. As shown in FIGS. 12A-13, the arm coupling portion 376 extends from the engagement portion 374 in a first direction 377 perpendicular from the arm coupling portion 376 and parallel to the axis 373, and the proximal arm 341 extends from arm coupling portion 376 in a second direction 378 perpendicular to the first direction 377. The second 45-degree joint part 372 further includes a rail coupling portion 381 coupled to (or comprising) the carriage 326 and coupled to the engagement portion 375.

The second proximal joint 330b includes a rotatable coupling between the first and second 45-degree joint parts 371 and 372 that allows for relative rotation therebetween about an axis 382. For example, as illustrated in FIG. 13, in some embodiments, the second proximal joint 330b includes a rotational bearing mechanism 383 that rotatably couples the first and second 45-degree joint parts 371 and 372 together. In some embodiments, the rotational bearing mechanism 383 has an outer bearing part 384 that is coupled to the first 45-degree joint part 371 and an inner bearing part 385 that is coupled to the second 45-degree joint part 372, with the inner and outer bearing parts 384 and 385 being rotatable relative to one another. For example, in some embodiments ball bearings are retained in a raceway between the inner and outer bearing parts 384 and 385 to facilitate low friction rotation. One of ordinary skill in the art would understand other types of bearing mechanisms that could be used as the rotational bearing mechanism 383, including plain bearings (with or without lubricants, coatings, or other friction reducing additives).

Due to the 45 degree angles of the engagement portions 374 and 375, rotation of the first 45-degree joint part 371 relative to the second 45-degree joint part 372 about the axis 382 causes the arm coupling portion 376 to change orientation relative to the second 45-degree joint part 372 and hence relative to the rail. In particular, in a first state illustrated in FIG. 12B, the arm coupling portion 376 extends vertically from the engagement portion 374 (i.e., the first direction 377 is vertical in this state), and in a second state illustrated in FIG. 12A, the arm coupling portion 376 extends horizontally from the engagement portion 374 (i.e., the first direction 377 is horizontal in this state). The state of FIG. 12A is reached from the state of FIG. 12B by rotating the first 45-degree joint part 372 180 degrees about the axis 382 and by rotating the proximal arm 341 about the first proximal joint 330a by 180 degrees. Thus, rotation about the second proximal joint 330b moves the proximal arm 341 through a range of motion between and including a vertical inclined orientation (FIG. 12A) and a horizontal orientation (FIG. 12B).

The second proximal joint 330b described above, including the first and second 45-degree joint parts 371 and 372, can be relatively compact as compared to other joints, and can also be stiffer than other joints because the axis 382 is not aligned with the other axes of manipulator and so compliance about axis 382 will not affect other axes. On the other hand, other joints, such as the second proximal joint 230b described above can allow more options for orientations of the proximal arm (e.g., any orientation between horizontal and vertical, in some embodiments), whereas in some circumstances the second proximal joint 330b can allow for a discrete set of fixed orientations (e.g., fully vertical and fully horizontal, in some embodiments).

The 45-degree angles of the joint parts 371 and 372 described above are examples only and other angles could be used in other embodiments. The angles used will determine the range of motion of the proximal arm 341. For example, if it is not needed to reach the full 90° of inclination for the proximal arm 341 in the final position, the joint parts 371 and 372 can be angled at less than 45°. For example, in some embodiments 70° inclination of the proximal arm 341 is sufficient to provide the desired reach across the table, and therefore the joint parts 371 and 372 are angled at 35°. Any other desired combination of angles of the joint parts 371 and 372 can be used in other embodiments to obtain a variety of angles of inclination of the proximal arm 341.

Turning now to FIG. 14, an embodiment of a wrist 447 will be described. The wrist 447 can be used as the wrists 147 or 247 described above, and duplicative description of similar aspects of the wrist 447 is omitted below.

The wrist 447 includes three rotational degrees of freedom of motion about axes 451, 453, and 454. More specifically, the degrees of freedom of motion provide for rotation of an instrument holding portion 469 coupled to the wrist 447 about the axes 451, 453, and 454. These axes 451, 453, and 454 are mutually orthogonal to one another, meaning that in a neutral state of the wrist, such as the state depicted in FIG. 14, the axis are all perpendicular to one another (however they are not necessarily all perpendicular in other states). In some embodiments, the axis 451 is orthogonal (perpendicular) to the axis 454 in all states, and the axis 454 is orthogonal (perpendicular) to the axis 453 in all states, but the axes 453 and 451 can be non-perpendicular two one another in certain states other than the neutral state. The degrees of freedom of motion of the wrist can be referred to as roll (i.e., rotation about axis 451), pitch (i.e., rotation about axis 454), and yaw (i.e., rotation about axis 453). In some embodiments, one or more of the degrees of freedom of motion of the wrist 447 is a redundant degree of freedom of motion (redundant degrees of freedom of motion being described above). For example, in some embodiments the pitch and/or yaw degrees of freedom of motion of the wrist 447 are redundant.

The wrist includes a first link 486a, a second link 486b, a first wrist joint 492, and a second wrist joint 493. The first link 486a is rotatably coupled to the distal end portion of a distal arm 443 (which can be used as the distal arm 143 or 243) of a manipulator via the first wrist joint 492 such that the first link 486a can rotate about the axis 454, which is perpendicular to a longitudinal dimension 487 of the distal arm 443 and perpendicular to axis 451 (described further below). Thus, the first wrist joint 492 provides for the pitch degree of freedom of motion for the wrist 447 described above. The second link 486b is rotatably coupled to the first link 486a via the second wrist joint 493 such that the second link 486b can rotate about the axis 453, which is perpendicular to the axis 454. Thus, the second wrist joint 493 provides for the yaw degree of freedom of motion of the wrist 447 described above. (The axis 453 is also perpendicular to the longitudinal dimension 487 of the distal arm 443 in a neutral state of the wrist 447, as depicted in FIG. 14.) The roll degree of freedom of motion of the wrist 447 can be provided by a third joint positioned proximally of the wrist. For example, in some embodiments the distal arm 443 includes a distal portion and a proximal portion that are rotatably coupled together by a rotational joint, such that the distal portion of the distal arm 443 can rotate relative to the proximal portion thereof about the axis 451 aligned with the longitudinal dimension 487. Because the wrist 447 is coupled to the distal portion of the distal arm 443, the wrist 447 rotates along with the distal portion of the distal arm 443 about the axis 451, thus providing the roll degree of freedom of motion. In other embodiments, the first link 486a of the wrist 447 is coupled to the distal arm 443 via a rotational joint (not illustrated) that provides for rotation between the first link 486a and the distal arm 443, thus providing the roll degree of freedom of motion. In still other embodiments, the distal arm 443 is rotatably coupled to another link assembly (e.g., intermediate link assembly 262) by a joint (not illustrated) that provides for rotation of the entire distal arm 443 (and hence the wrist 447 coupled thereto) about the axis 451, thus providing for the roll degree of freedom of motion.

In some embodiments, the first and second rotational joints 492 and 493 are actuatable by actuators (e.g., motors) disposed externally from the wrist, such as in a more proximal portion of the manipulators (e.g., in the distal arm 443). The first and second rotational joints 492 and 493 can be coupled to the actuators by actuation elements (not illustrated), such as cables, filaments, belts, bands, linkages, etc., which extend distally from the more proximal portion of the manipulator into the wrist 447. Housing the actuators remotely from the wrist 447 allows the wrist 447 to have a relatively small diameter, and also reduces the moment arm of the manipulator.

As shown in FIG. 14, an instrument holding portion 469 coupled to the wrist 447 (which can be used as instrument holding portion 269) includes an instrument holder base member 455 and an accessory mount portion 456 are coupled to and extend in different directions from the second link 486b. An insertion axis 490 is configured to pass through an accessory (e.g., cannula) mounted to the accessory mount portion 456, with the insertion axis 490 being an axis along which a shaft of an instrument extends when mounted to an instrument mount (not illustrated) coupled to the instrument holder base member 455. As shown in FIG. 14, the insertion axis 490 is offset from the wrist 447, in particular offset from the axis 453, by a predetermined distance. In some embodiments, the axes 490 and 453 are arranged in the same plane as one another (a first plane). Furthermore, the axes 453 and 451 are arranged in the same plane as one another (a second plane). In some states, the first and second planes are parallel, while in other states the first and second planes are angled relative to one another (depending on rotation about the axis 453). In addition, the axes 454 and 451 are arranged in the same plane as one another (a third plane). Moreover, the axes of rotation 451, 453, and 454 do not intersect with a remote center of motion of the system. In addition, in the embodiment illustrated in FIG. 14, a longitudinal dimension of the instrument holder base member 255 is offset slightly from the axis 453. In other embodiments (not illustrated), the longitudinal dimension of the instrument holder base member 255 can be aligned with the axis 453.

Turning now to FIGS. 15-24, another embodiment of a table-mounted manipulator system 500 (“system 500”) is described below. The system 500 can be used as (i.e., is one example configuration of) the system 100, and some components of the system 500 can be used as (i.e., are example configurations of) components of the system 100 described above. In particular, the system 500 includes manipulators 540 (e.g., manipulators 540_1, 540_2, 540_3) that can be used as the manipulators 140 of the system 100. Thus, the descriptions of the components of the system 100 above are applicable to the related components of the system 500, and duplicative descriptions of these components are omitted below. The related components of the systems 100 and 500 are given reference numbers having the same right-most two digits—for example, 140 and 540. Although the system 500 is one embodiment of the system 100, the system 100 is not limited to the system 500.

As shown in FIG. 15, the system 500 includes a table assembly 501, two rail assemblies 520 coupled to the table assembly 501, and multiple manipulators 540 coupled to the rail assemblies 520. Each manipulator 540 is configured to support one or more instruments (not illustrated), which can be removably or permanently mounted thereon. The system 500 also can include a control system (not illustrated), a user input and feedback system (not illustrated), and/or an auxiliary system (not illustrated) similar to those described above in relation to the system 100. In some embodiments, the system 500 is configured as a computer-assisted, teleoperable medical system. In other embodiments, the system 500 is configured as a teleoperable system for use in non-medical contexts.

As shown in FIG. 1, the table assembly 501 includes a platform assembly 510 configured to support the patient or inanimate workpiece, a support column 502 coupled to and supporting the platform assembly 510, and a base 505 coupled to the support column 502. The base 505 can be configured to contact the ground or other surface upon which the table assembly 501 rests, and in some embodiments the base 505 includes wheels 506 to allow movement of the system 500 along the ground or other surface. In some embodiments the support column 502 includes a telescoping support column that can raise or lower the platform assembly 510.

The platform assembly 510 includes multiple platform sections configured to support the patient or workpiece, which can be fixed or movable relative to another and/or the support column 502. In addition to moving individual platform section, the platform assembly 510 as a whole can be movable relative to the support column 502 through one or more degrees of freedom, which can include both translational and rotational degrees of freedom. For example, in some embodiments the platform assembly 510 is configured similarly to the platform assembly 210 described above.

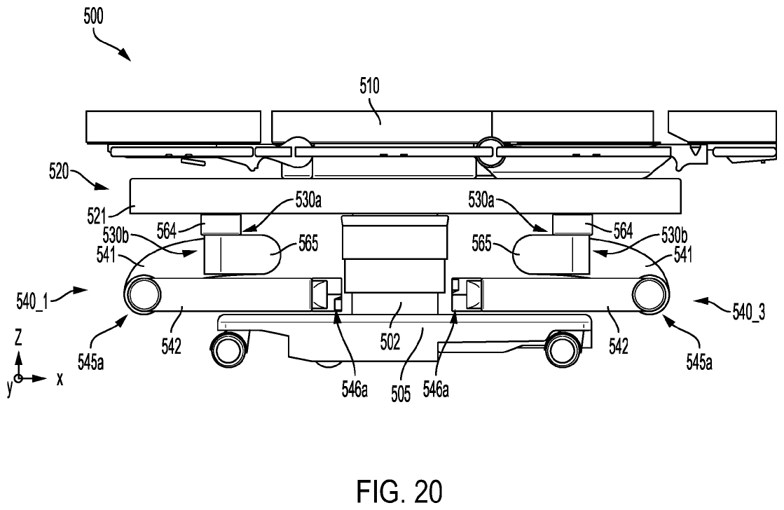

As shown in FIGS. 15 and 20, the manipulators 540 are coupled to the table assembly 501 via the rail assemblies 520. One rail assembly 520 is provided for each of the two longitudinally extending sides of the platform assembly 510. As shown in FIGS. 15 and 20, the rail assembly 520 includes a movable rail 521 (“rail 521”), first carriages 526 coupling the manipulators 540 to the rail 521, and one or more second carriages 527 coupling the rail 521 to the table assembly 501. Each first carriage 526 couples a respectively corresponding one of the manipulators 540 to the rail 521 such that the manipulators 540 can translate relative to the rail 521 along a longitudinal dimension 597 of the rail 521. In some examples, the first carriage 526 can be part of the corresponding manipulator 540. For example, in some embodiments the first carriage 526 includes a track or other engagement feature that is coupled to (or is part of) a top surface of a first proximal joint housing 564 (described below) of the manipulator 540 and configured to engage with complementary engagement features of the rail 521 to movably couple the manipulator 540 to the rail 521. In addition, each second carriage 527 couples the rail assembly 520 to the table assembly 501 (e.g., either to the platform assembly 510 or to the support column 502) such that the rail 521 can translate relative to the table assembly 501 along a longitudinal dimension of the rail 521. For example, in some embodiments the second carriage 527 includes a track or other engagement feature that is coupled to (or is part of) the table assembly 501 and configured to engage with complementary engagement features of the rail 521 to movably couple the manipulator the rail 521 to the table assembly 501.

In some embodiments, the rail assembly 520 further includes translation mechanisms (not illustrated) configured to drive translation of the first carriages 526 relative to the rail and to drive translation of the rail 521 relative to the second carriage 527. The translation mechanisms can include actuators, such as electrical motors or other actuation devices (e.g., hydraulic, pneumatic, or other devices to provide a motive force).

As noted above, the system 500 includes multiple manipulators 540. In some embodiments, four manipulators 540 are provided, with two coupled to each rail assembly 520 (of these four manipulators 540, only manipulators 540_1, 540_2, and 540_3 are visible in the figures). In other embodiments, more or fewer manipulators 540 can be used, such as one, two, three, five or more manipulators. In addition, more or fewer manipulators 540 can be coupled to the rail assemblies 520, and the rail assemblies 520 do not necessarily have to have the same number of manipulators 540 as each other.

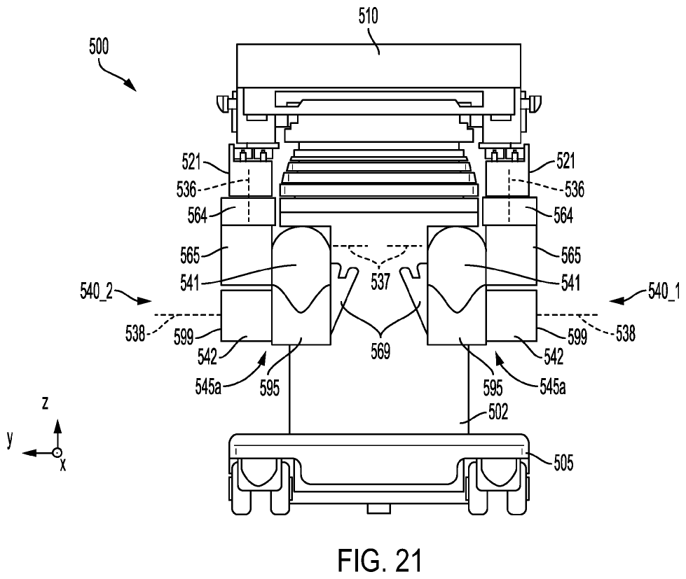

In FIG. 15, the manipulators 540 are shown in deployed states or configurations, whereas in FIGS. 21-24 the manipulators 540 are shown in a stowed state under the platform assembly 510 while remaining coupled to the rail assembly 520 (not all of the manipulators 540 are visible in each of the Figures). The deployed states include states in which one or more of the manipulators 540 are not stowed, which in some embodiments means at least that the one or more manipulators 540 are at least partially unfolded/uncompacted while remaining coupled to the rail assembly 520, and removed from a stowed location, e.g., removed out from under the platform assembly 510. The manipulators 540 can be positioned in a variety of deployed states, which can include a variety of configurations and positions of the manipulators 540 including but not limited to those shown in FIG. 15.

The manipulators 540 include a number of links moveably coupled together via joints, as described above in relation to the manipulators 140 and 240. In particular, FIG. 16 illustrates one of the manipulators 540 in isolation. As shown in FIGS. 15 and 16, each manipulator 540 includes a proximal link assembly 561 including a proximal arm 541 coupled to the rail assembly 520 via one or more proximal joints 530 and a carriage 526, an intermediate link assembly 562 including an intermediate arm 542 coupled to a distal end portion of the proximal link assembly 561 via one or more intermediate joints 545, and a distal link assembly 563 including a distal arm 543 coupled to the intermediate link assembly 562 via one or more distal joints 546. The distal link assembly 563 also includes an instrument holding portion 569 coupled to the distal arm 543 and configured to support the instrument 550.

As noted above, the proximal link assembly 561 includes a proximal arm 541 and proximal joints 530. A proximal end portion of the proximal arm 541 of each manipulator 540 is coupled to the rail assembly 520 via the proximal joints 530. Specifically, the proximal link assembly 561 includes a first proximal joint housing 564, a second proximal joint housing 565, and a third proximal joint housing 594 (which is coupled to or a part of the proximal arm 541), with the rail assembly 520 being coupled to the first proximal joint housing 564 (via first carriage 526), first proximal joint housing 564 being rotatably coupled to the second proximal joint housing 565, and second proximal joint housing 565 being rotatably coupled to the third proximal joint housing 594 (and hence to the proximal arm 541).

The rotatable coupling between first proximal joint housing 564 and the second proximal joint housing 565 forms a first proximal joint 530a. The first proximal joint 530a allows for rotation of the proximal arm 541 relative to the rail 521 about a first axis 536 (via rotation between first proximal joint housing 564 and second proximal joint housing 565). The first axis 536 is perpendicular a longitudinal and lateral dimensions of the rail 521 (i.e., the first axis 536 extends parallel to the z-axis in FIGS. 15 and 20), or in other words perpendicular to both a longitudinal dimension and a lateral dimension of the platform 510 (in a neutral position of the table assembly 501).

The rotatable coupling between the second proximal joint housing 565 and third proximal joint housing 594 forms a second proximal joint 530b The second proximal joint 530b provides for rotation of the proximal arm 541 relative to the rail 521 around a second axis 537 orthogonal to the first axis 536 and parallel to a longitudinal dimension of the rail 521 (i.e., the second axis 537 extends parallel to the x-axis in FIGS. 15 and 20). Rotation of the proximal arm 541 around the second axis 537 (e.g., via second proximal joint 530b) causes the proximal arm 541 to incline or decline relative to a horizontal plane (e.g., a plane parallel to the longitudinal or lateral dimensions of the platform 510 in embodiments in which the rail assembly 520 is coupled to the platform 510, or a plane parallel to the ground in embodiments in which the rail assembly 520 is coupled to the support column 502), thus raising or lowering a distal end of the proximal arm 541 relative to the rail 521. As the proximal arm 541 inclines relative to the horizontal plane, movement of the proximal arm 541 can cause more distal portions of the manipulator to correspondingly both raise and extend further across the table (as opposed to vertical movement alone).

As shown in FIGS. 15 and 16, a distal end portion of the proximal arm 541 includes (or is coupled to) a first intermediate joint housing 595, and a proximal end portion of the intermediate arm 542 includes (or is coupled to) a second intermediate joint housing 599, with the first and second intermediate joint housings 595 and 599 being rotatably coupled together. The rotatable coupling between first and second intermediate joint housings 595 and 599 forms a first intermediate joint 545a. The first intermediate joint 545a allows the intermediate arm 542 to rotate relative to the proximal arm 541 about a third axis 538, which is parallel to the second axis 537.

In addition, in some examples the intermediate arm 542 includes a first link 542a and a second link 542b, which are translatable and/or rotatable relative to one another. More specifically, in some examples, the first and second links 542a and 542b are translatable relative to one another along a direction 549 parallel to a longitudinal dimension of the intermediate arm 542 and/or rotatable relative to one another about a fourth axis 539 that is parallel to the longitudinal dimension of the intermediate arm 542. The translatable and/or rotatable coupling between first link 542a and a second link 542b forms a second intermediate joint 545b. In some embodiments, the second intermediate joint 545b provides for just rotation, in other embodiments the second intermediate joint 545b provides for just translation, and in still other embodiments second intermediate joint 545b provides for both rotation and translation.

The distal link assembly 563 includes a distal arm 543, a wrist 547, and an instrument holding portion 569 coupled to the distal arm 543 via the wrist 547. As shown in FIGS. 15 and 16, a proximal end portion of the distal arm 543 of each manipulator 540 is rotatably coupled to the second link 542b of the intermediate arm 542 via a first distal joint 546a. More specifically, a distal end of the second link 542b is coupled to or includes a first distal joint housing 567, which is rotatably coupled to a second distal joint housing 568 that is coupled to or part of the distal arm 543. The first distal joint 546a allows for rotation of the distal arm relative to the intermediate arm 542 about a fifth axis 552, which is perpendicular to the longitudinal dimension of the intermediate arm 542 and the longitudinal dimension of the distal arm 543. In addition, a second distal joint 546b rotatably couples the wrist 547 to the second distal joint housing 568 (via the arm 543) such that the wrist 547 can rotate relative to the second distal joint housing 568 about a sixth axis 551, which is parallel to the longitudinal dimension of the distal arm 543. Rotation about this sixth axis 551 via the second distal joint 546b constitutes a degree of freedom of motion of the wrist 547, which can be referred to as roll. Thus, the sixth axis 551 can also be called a roll axis. In some embodiments, a distal end portion of the distal arm 543 moves along with the wrist 547 relative to a proximal end portion of the distal arm 543 as the wrist rotates around the sixth axis 551.

In addition to the roll degree of freedom of motion described above, the wrist 547 allows for rotation of the instrument holding portion 569 relative to the arm 543 about two additional axes, the seventh and eighth axes 553 and 554. The seventh and eight axes 553 and 554 are perpendicular to one another. The seventh and eight axes 553 and 554 are also perpendicular to the sixth axis 551 and hence also to the longitudinal dimension of the distal arm 543 in a neutral state of the wrist 547, but not necessarily in other states of the wrist 547. Specifically, in some embodiments, the seventh axis 553 remains perpendicular to the sixth axis 551 in all states of the wrist, whereas the eight axis 554 does not. In other embodiments, this relationship is reversed, with the eight axis 554 remaining perpendicular to the sixth axis 551 while the seventh axis 553 does not. Rotation about the seventh and eighth axes 553 and 554 can be referred to as pitch and yaw degrees of freedom of motion, respectively, and thus the seventh and eighth axes 553 and 554 can be referred to as pitch and yaw axes, respectively. In particular, the wrist 547 includes two wrist joints that provide for rotation about the seventh and eighth axes.

Some of the degrees of freedom of motion of the manipulators 540 described above are redundant in the sense of not being strictly necessary in order to manipulate the instrument, but inclusion of these redundant degrees of freedom of motion can improve manipulation of the manipulators 540 in some circumstances, for example by helping with reach and collision avoidance. For example, the rotation of the wrist 547 about the eighth axis 554 (yaw), the extension of the proximal arm 541 along direction 548, and the extension of the intermediate arm 542 along the direction 549 can be redundant degrees of freedom of motion. In some embodiments, one, some, or all of these degrees of freedom of motion are omitted. In some embodiments, some or all of these redundant degrees of freedom of motion can be used as setup joints whose position is fixed so as to obtain a desired deployed pose of the manipulator 540 but which thereafter remain stationary during the procedure (or are only moved infrequently during the procedure when a change of pose is desired).

As shown in FIGS. 16, the instrument holding portion 569 includes a instrument holder base member 555 coupled to the wrist 547 and extending parallel to the eighth axis 554, an instrument holder 544 movably coupled to the instrument holder base member 555, and an accessory mount portion 556 coupled to one end portion of the instrument holder base member 555. The instrument holder 544 is translatable along a length of the instrument holder base member 555 along a direction parallel to the eighth axis 554. The instrument holder 544 includes an interface to couple to an instrument 550 mounted thereto. For example, the interface can include output couplers (not illustrated) to engage (directly or indirectly via an intermediary) with input couplers (not illustrated) of the instrument 550 to provide driving forces or other inputs to the mounted instrument 550 to control various degree of freedom movement and/or other functionality of the instrument 550. The accessory mount portion 556 is configured to receive an accessory mounted thereon, such as a cannula (a cannula is illustrated mounted to the access mount portion 556 in FIG. 16 as an example). The cannula mounted to the accessory mount portion 556 can be positioned to receive an instrument shaft of an instrument 550 mounted to the instrument holder 544. The instrument shaft and a passage through the cannula can define an insertion axis along with the instrument can translate in response to translation of the instrument holder 544 along the instrument holder base member 555. A remote center of motion can be located on the insertion axis in, at, or near the cannula.

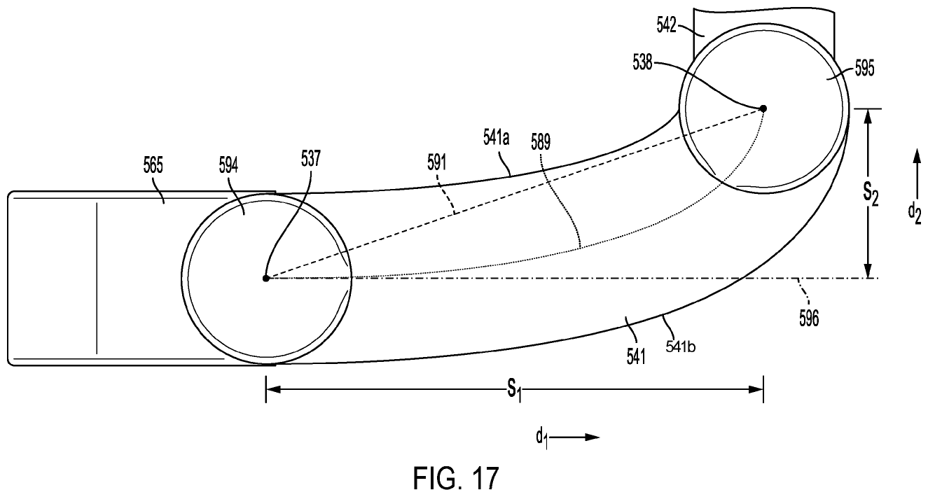

As shown in FIGS. 15-17, the proximal arm 541 of the manipulator 540 has an asymmetrical shape. An asymmetrical shape means that, in extending from a proximal end portion of the proximal arm 541 (e.g., the third proximal joint housing 594) to a distal end portion of the proximal arm 541 (e.g., the first intermediate joint housing 595), the proximal arm 541 follows a path that deviates from a hypothetical straight line extending between (i.e., connecting) the two end portions. More specifically, as shown in FIG. 17, a centerline 589 of the proximal arm 541, while extending between the second axis of rotation 537 of the second proximal joint 530b and the third axis of rotation 538 of the first intermediate joint 545a, deviates from a straight line 591 between these two axes of rotation 537 and 538. In other words, the respective axes of rotation 537 and 538 (which are parallel to one another) are offset from one another along both a first direction d1 and a second direction d1, as indicated by offsets s1 and s2 in FIG. 17. The first direction d1, which corresponds to offset s1, is parallel to the line 596, which is tangent to the centerline 589 of the proximal arm 541 at the second axes of rotation 537. This line 596 corresponds to an initial direction of extent of the proximal arm 541 as initially extends from the second rotational axis 537. The second direction d2, which corresponds to offset s2, is perpendicular to the first direction d1.

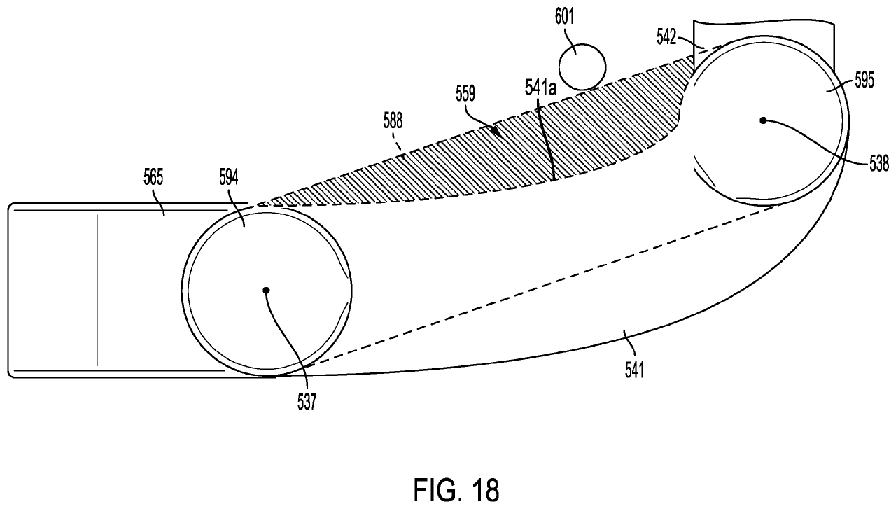

As shown in FIG. 17, the asymmetrical shape of the proximal arm 541 results in the proximal arm 541 having a concave side 541a and a convex side 541b. As used herein, concave and convex are judged relative to the platform assembly 510 in a state of the proximal arm 541 being oriented approximately vertically (i.e., with line 596 parallel to the z-axis). Consequently, as shown in FIG. 18, there is an open space 559 along the concave side 541a of the proximal arm 541, which would otherwise have been occupied by the proximal arm if the proximal arm were to extend in a straight line between the axes 537 and 538. As shown in FIG. 18, this open space 559 corresponds to the region between line 588 and an outer surface of the proximal arm 541. The line 588 in FIG. 18 indicates where an edge of the proximal arm would have been located if the proximal arm were to extend in a straight line between the axes 537 and 538, instead of having the asymmetrical shape. This open space 559 can allow the manipulator 540 to be placed in poses that otherwise would not be possible with a straight proximal arm of similar size. For example, if the proximal arm 541 is rotated upwards and towards the platform assembly 510, a straight proximal arm will need to be stopped at a given point to avoid a collision between the arm and the patient, the platform assembly 510, or other objects, whereas the proximal arm 541 disclosed herein can be able to continue rotating some distance beyond that given point because the patient, table, or other object, which would have otherwise collided with the straight arm, can instead fit within the open space 559 along the concave side 541a of the proximal arm 541. For example, if a hypothetical object 601 were located adjacent the proximal arm as shown in FIG. 18, then a straight proximal arm would need to stop rotating toward the object 601 at the point illustrated in FIG. 18 (to avoid collision therewith), whereas the asymmetrical proximal arm 541 could continue rotating toward the object 601 beyond the point illustrated in FIG. 18 because of space afforded by the open space 559. This additional range of rotation of the proximal arm 541 can allow for intermediate joint 542 to be raised higher and/or brought farther inward towards the centerline of the platform assembly 510, which can allow for the manipulator 540 to reach farther across the platform assembly 510. Moreover, when the manipulators 540 are stowed, the open space 559 can allow for a more compact pose of the manipulator 540, as is described in greater detail below.

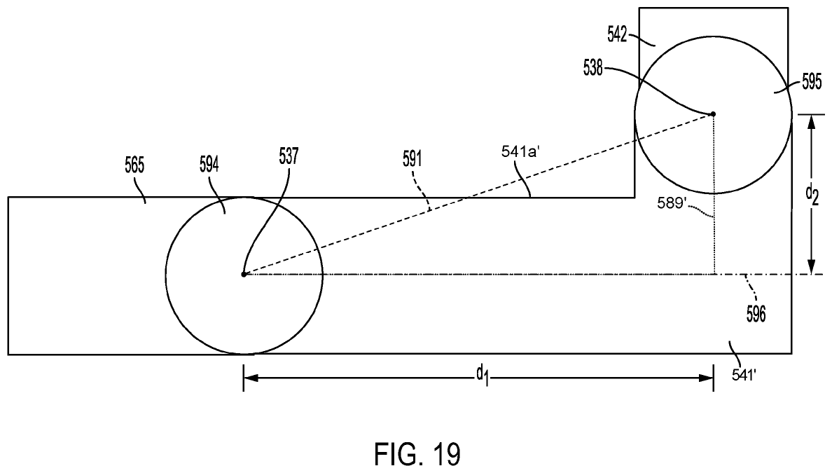

FIGS. 15-18 and 20-24 illustrated an example embodiment in which the asymmetrical shape of the proximal arm 541 is a smoothly curved shape, like an arc. However, in other embodiments, the proximal arm 541 can have other asymmetrical shapes that satisfy the criteria described above, including smoothly curved shapes that follow a different curve than the one illustrated, such as a curve including: a portion of a circle, a portion of an ellipse, a portion of a parabola, a portion of a hyperbola, or any other smooth curve. As another example, in some embodiments the proximal arm has a segmented shape including multiple straight and/or curved segments joined together at angles. For example, FIG. 18 illustrates an embodiment of a proximal arm 541′, which is a variation of the proximal arm 540 in which the asymmetrical shape of the proximal arm 541′ includes an L-shape with two straight segments coupled together at a right angle. As can be seen in FIG. 19, the centerline 589′ of the proximal arm 541′ deviates from the line 591, as described above in relation to proximal arm 541. Although concave and convex are often associated with curves, as used herein the terms concave and convex also are applicable to segmented shapes with straight segments, such as the L-shape shown in FIG. 19. Thus, the proximal arm 541′ has a concave side 541a′ and a convex side 541b′.

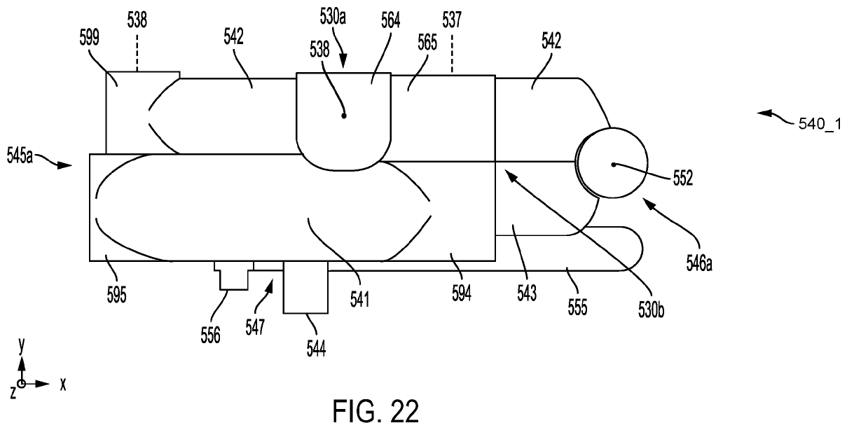

FIGS. 20-24 illustrate the manipulators 240 in a stowed state (note that other stowed states besides those illustrated are possible). As shown in FIGS. 20 and 21, in the illustrated stowed state the manipulators 240 are positioned beneath the platform assembly 210 in a compact folded configuration. As shown in FIGS. 20 and 21, in this stowed state the first and second proximal joint housings 564 and 565 are positioned directly below the rail 521. Moreover, as shown in FIGS. 20-24, the proximal arm 541 is positioned laterally adjacent to, and inward of, the second proximal joint housing 565. The intermediate arm 542 is positioned laterally adjacent to, and outward of, the proximal arm 541. Moreover, as shown in FIGS. 20-24, the intermediate arm 542 is positioned directly below the second proximal joint housing 565 (and the rail 521). As shown in FIGS. 22-24, the distal arm 543 is positioned laterally adjacent to, and inward of, the intermediate arm 542. In addition, the distal arm 543 is parallel to the intermediate arm 542 (i.e., their respective longitudinal axes are parallel), or in other words, the distal arm 543 and the intermediate arm 542 are aligned and at least partially overlap along a direction parallel to the lateral dimension of the platform assembly 510 (e.g., along the y-axis). Furthermore, as shown in FIGS. 22-24, at least part of the distal arm 543 is positioned directly below the proximal arm 541, with the distal arm 543 extending into the open space 559 along the concave side of the proximal arm 541 (i.e., the proximal arm 541 and the distal arm 543 at partially overlap along a direction parallel to a height dimension of the platform assembly 510, or in other words along the z-axis). The instrument holding portion 569 is laterally adjacent to the distal arm 543, with the instrument holder base member 555 extending parallel to the distal arm 543. In addition, a portion of the instrument holder base member 555 and wrist 547 is positioned below the proximal arm 541. As shown in FIG. 22, the instrument holding portion 569 can protrudes somewhat laterally inward relative to an edge of the proximal arm 541, particularly portions of the instrument holder 544 and the accessory mount portion 556.

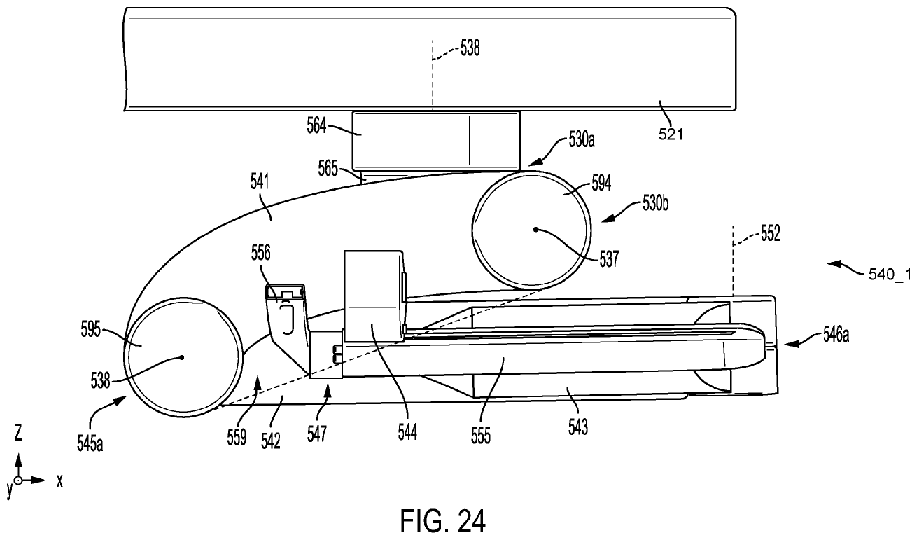

The stowed pose of the manipulator 540 described above is very compact. If the proximal arms 541 were straight rather than being asymmetrically shaped, this compact pose can not be possible. For example, as shown in FIG. 24, the distal arm 543 and the instrument holding portion 569 extend into the open region 559, but if the distal arm 543 were straight this would not be possible. Thus, to avoid collision with a straight distal arm, the distal arm 543 can need to be angled inward so that the distal end of the distal arm 543 is laterally adjacent to the proximal arm 541, resulting in the distal arm 543 being at an angle relative to the intermediate arm 542 instead of parallel thereto. But this configuration is less compact. Other stowed configurations could be used with a straight distal arm to avoid the collisions, but these other stowed configurations similarly require changes to above-described arm poses which result in a less compact shape. Thus, the asymmetrical shape of the proximal arm 541 allows for a stowed configuration that is much more compact than can otherwise be possible.

In embodiments, the manipulators 540 can be selectively deployable in two different deployed configurations, including a first deployed configuration in which the proximal arm 541 is in a concave orientation and a second deployed configuration in which the proximal arm 541 is in a convex orientation. These configurations can be referred to herein as a concave configuration and a convex configuration. The concave orientation of the proximal arm 541 refers to an orientation in which the concave side 541a′ faces generally upward if the proximal arm 541 is extending horizontally away from the table assembly 501. Moreover, in the concave orientation the second proximal joint housing 565 extends generally outward from the rail (i.e., away from a centerline of the table assembly 501). Conversely, the convex orientation of the proximal arm 541 refers to an orientation in which the concave side 541a′ faces generally downward if the proximal arm 541 is extending horizontally away from the table assembly 501. Moreover, in the convex orientation the second proximal joint housing 565 extends generally inward from the rail (i.e., toward a centerline of the table assembly 501). For example, FIG. 15 illustrates the manipulator 540_1 in the concave configuration and manipulator 540_2 in the convex configuration.

In some embodiments, the manipulators 540 can be transitioned between the concave configuration and the convex configurations. For example, when a manipulator 540 is moved from an stowed configuration to a deployed configuration, the manipulator 540 can be selectively deployed in either the concave configuration or the convex configuration as desired. From the stowed configuration, the second proximal joint housing 565 is rotated to face outward (away from the table assembly 501) and then the proximal arm 541, intermediate arm 542 and distal arms 543 are unfolded to achieve the concave configuration. To achieve the convex configuration, from the stowed configuration the second proximal joint housing 565 is rotated to face inward (away the table assembly 501) and then the proximal arm 541, intermediate arm 542 and distal arms 543 are unfolded.

These concave and convex configurations of the manipulator 540 can provide increased flexibility for the user in obtaining desired poses, as some poses that might not be difficult or not possible with one configuration can be possible or easier with the other, and vice versa. For example, in the concave configuration of the manipulator 540 (with the concave orientation of proximal arm 541), the manipulator 540 can be able to reach farther across the platform assembly 510. As another example, in the convex configuration of the manipulator 540 (with the convex orientation of the proximal arm 541), the manipulator 540 can be able to position the proximal end portion of the intermediate arm 542 lower and farther inward, which can allow the instrument holding portion 569 to be angled farther backward relative to the distal arm 543 when utilizing entry ports that are located low on a patient on a same side thereof as the manipulator 540.

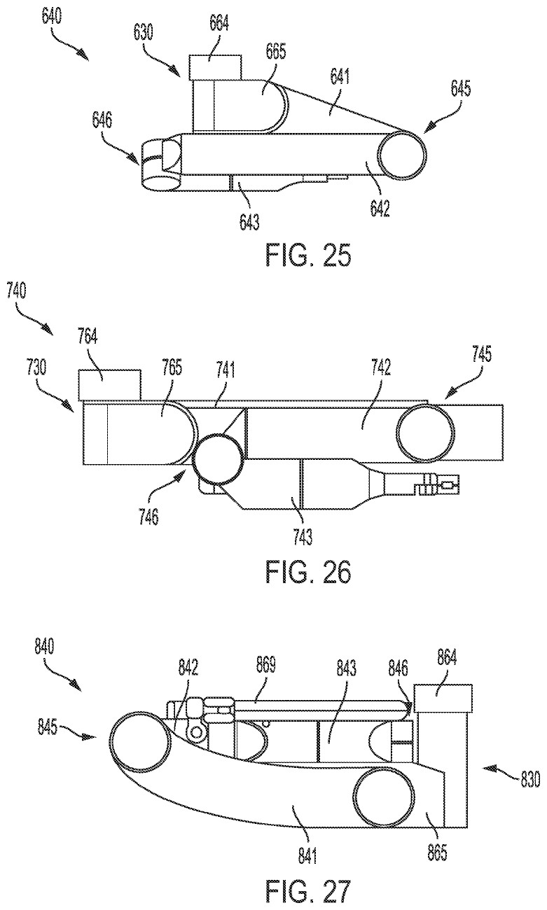

Turning now to FIGS. 25-27, some additional embodiments of manipulator arms 640, 740, and 840 are illustrated. FIGS. 25-27 show these manipulator arms 640, 740, and 840 in example stowed positions. The manipulator arms 640, 740, and 840 are configurations of the manipulators 140, and include a number of links moveably coupled together via joints, as described above in relation to manipulator arms 140, 240, and 540. Elements of the manipulator arms 640, 740, and 840 that are similar to elements described above in relation to manipulator arms 140 and/or 540 are given similar reference numbers (with the same last two digits), and duplicative description thereof is omitted below.

FIG. 25 illustrates an example manipulator arm 640. The manipulator arm 640 is similar to the manipulator arm 540 described above except that the manipulator arm 640 has a straight proximal arm 641, unlike the asymmetrical proximal arm 541 of the manipulator arm 540. The manipulator arm 640 also includes a first proximal joint housing 664 configured to be coupled to a rail, a second proximal joint housing 665 coupled to the first proximal joint housing 664 and to the proximal arm 641, with the first and second proximal joint housings 664, 665 forming proximal joints 630. The manipulator arm 640 also includes an intermediate arm 642 coupled to a distal end portion of the proximal arm 641 via one or more intermediate joints 645, a distal arm 643 coupled to the intermediate arm 642 via one or more distal joints 646, and an instrument holding portion (not visible) coupled to the distal arm 643. As shown in FIG. 25, the manipulator arm 640 can be stowed in a similar fashion as the manipulator arm 540. However, because proximal arm 641 is straight, the stowed configuration of the manipulator arm 640 is not as compact as that of the manipulator arm 540. For example, the distal arm 643 can protrude farther downward than the intermediate arm 643 does, so that the distal arm 643 and the intermediate arm 643 are not aligned horizontally with one another. This can be needed to prevent interference between the distal arm 643 and the proximal arm 641. In contrast, in the manipulator arm 540 such interference is avoided due to the asymmetrical shape of the proximal arm 541, and therefore the distal arm 543 can be positioned horizontally aligned with the intermediate arm 542.

With reference to FIG. 26, the manipulator arm 740 is similar to the manipulator arm 640 described above except that the manipulator arm 740 has an extendable straight proximal arm 741. That is, the proximal arm 741 can include two parts that can translate (and in some cases, also rotate) relative to one another, like the proximal arm 241 described above. The manipulator 740 also includes a first proximal joint housing 764 coupled to a rail, a second proximal joint housing 765 coupled to the first proximal joint housing 764 and to the proximal arm 741, with the first and second proximal joint housings 764, 765 forming proximal joints 730. The manipulator 740 also includes an intermediate arm 742 coupled to a distal end portion of the proximal arm 741 via one or more intermediate joints 745, a distal arm 743 coupled to the intermediate arm 742 via one or more distal joints 746, and an instrument holding portion coupled to the distal arm 743. As shown in FIG. 26, the manipulator arm 740 can be stowed in a configuration in which the proximal arm 741 and intermediate arm 742 are horizontally aligned, with the distal arm 743 positioned below and vertically aligned with the intermediate arm 742.

FIG. 27 illustrates a manipulator arm 840 that is similar to the manipulator arm 540 described above except that the manipulator arm 840 has an extension 899 coupled to the second proximal joint housing 865 such that the second proximal joint housing 865 is positioned lower than the second proximal joint housing 565. In addition, a proximal arm 841 of the manipulator 840 is longer than the proximal arm 541. The manipulator arm 840 also includes a first proximal joint housing 864 configured to be coupled to a rail, the second proximal joint housing 865 coupled to the first proximal joint housing 864 and to the proximal arm 841, with the first and second proximal joint housings 864, 865 forming proximal joints 830. The manipulator arm 840 also includes an intermediate arm 842 coupled to a distal end portion of the proximal arm 841 via one or more intermediate joints 845, a distal arm 843 coupled to the intermediate arm 842 via one or more distal joints 846, and an instrument holding portion 869 coupled to the distal arm 843. As shown in FIG. 27, the manipulator arm 840 can be stowed in a manner that is reversed relative to how the manipulator arm 540 is stored. That is, instead of the proximal arm 841 curving downward like the proximal arm 541, the proximal arm 841 curves upward. Moreover, the distal arm 843 is positioned above the proximal arm 841 instead of below. But similar to the manipulator 540, the distal arm 843 is horizontally aligned with the intermediate arm 842.

The embodiments described herein can be well suited for use in any of a variety of medical procedures, as described above. Such procedures could be performed, for example, on human patients, animal patients, human cadavers, animal cadavers, and portions or human or animal anatomy. Medical procedures as contemplated herein include any of those described herein and include, for non-surgical diagnosis, cosmetic procedures, imaging of human or animal anatomy, gathering data from human or animal anatomy, training medical or non-medical personnel, and procedures on tissue removed from human or animal anatomies (without return to the human or animal anatomy). Even if suitable for use in such medical procedures, the embodiments can also be used for benchtop procedures on non-living material and forms that are not part of a human or animal anatomy. Moreover, some embodiments are also suitable for use in non-medical applications, such as industrial robotic uses, and sensing, inspecting, and/or manipulating non-tissue work pieces. In non-limiting embodiments, the techniques, methods, and devices described herein can be used in, or can be part of, a computer-assisted surgical system employing robotic technology such as the da Vinci® Surgical Systems commercialized by Intuitive Surgical, Inc., of Sunnyvale, California. Those skilled in the art will understand, however, that aspects disclosed herein can be embodied and implemented in various ways and systems, including manually operated instruments and computer-assisted, teleoperated systems, in both medical and non-medical applications. Reference to the daVinci® Surgical Systems are illustrative and not to be considered as limiting the scope of the disclosure herein.

As used herein and in the claims, terms such as computer-assisted manipulator system, teleoperable manipulator system, or the like should be understood to refer broadly to any system including one or more controllable kinematic structures (“manipulators”) that are movable and controllable at least in part through the aid of an electronic controller (with or without human inputs). Such systems can occasionally be referred to in the art and in common usage as robotically assisted systems or robotic systems. Such systems include systems that are controlled by a user (for example through teleoperation), by a computer automatically (so-called autonomous control), or by some combination of these. In examples in which a user controls at least some of the operations of the manipulator, an electronic controller (e.g., a computer) can facilitate or assist in the operation. The term “computer” as used in “computer-assisted manipulator systems” refers broadly to any electronic control device for controlling, or assisting a user in controlling, operations of the manipulator, and is not intended to be limited to things formally defined as or colloquially referred to as “computers.” For example, the electronic control device in a computer-assisted manipulator system could range from a traditional “computer” (e.g., a general-purpose processor plus memory storing instructions for the processor to execute) to a low-level dedicated hardware device (analog or digital) such as a discrete logic circuit or application specific integrated circuit (ASIC), or anything in between. Further, manipulator systems can be implemented in a variety of contexts to perform a variety of procedures, both medical and non-medical. Thus, although some examples described in greater detail herein can be focused on a medical context, the devices and principles described herein are also applicable to other contexts, such as industrial manipulator systems.

It is to be understood that both the general description and the detailed description provide example embodiments that are explanatory in nature and are intended to provide an understanding of the present disclosure without limiting the scope of the present disclosure. Various mechanical, compositional, structural, electrical, and operational changes can be made without departing from the spirit and scope of this description and the claims. In some instances, well-known circuits, structures, and techniques have not been shown or described in detail in order not to obscure the embodiments. Like numbers in two or more figures represent the same or similar elements.

Further, the terminology used herein to describe aspects of the invention, such as spatial and relational terms, is chosen to aid the reader in understanding example embodiments of the invention but is not intended to limit the invention. For example, spatial terms-such as “beneath”, “below”, “lower”, “above”, “upper”, “proximal”, “distal”, “up”, “down”, and the like—can be used herein to describe directions or one element's or feature's spatial relationship to another element or feature as illustrated in the figures. These spatial terms are used relative to the figures and are not limited to a particular reference frame in the real world. Thus, for example, the direction “up” in the figures does not necessarily have to correspond to an “up” in a world reference frame (e.g., away from the Earth's surface). Furthermore, if a different reference frame is considered than the one illustrated in the figures, then the spatial terms used herein can need to be interpreted differently in that different reference frame. For example, the direction referred to as “up” in relation to one of the figures can correspond to a direction that is called “down” in relation to a different reference frame that is rotated 180 degrees from the figure's reference frame. As another example, if a device is turned over 180 degrees in a world reference frame as compared to how it was illustrated in the figures, then an item described herein as being “above” or “over” a second item in relation to the Figures would be “below” or “beneath” the second item in relation to the world reference frame. Thus, the same spatial relationship or direction can be described using different spatial terms depending on which reference frame is being considered. Moreover, the poses of items illustrated in the figure are chosen for convenience of illustration and description, but in an implementation in practice the items can be posed differently.

In addition, the singular forms “a”, “an”, and “the” are intended to include the plural forms as well, unless the context indicates otherwise. And, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled can be electrically or mechanically directly coupled, or they can be indirectly coupled via one or more intermediate components, unless specifically noted otherwise. Mathematical and geometric terms are not necessarily intended to be used in accordance with their strict definitions unless the context of the description indicates otherwise, because a person having ordinary skill in the art would understand that, for example, a substantially similar element that functions in a substantially similar way could easily fall within the scope of a descriptive term even though the term also has a strict definition.

Elements and their associated aspects that are described in detail with reference to one embodiment can, whenever practical, be included in other embodiments in which they are not specifically shown or described. For example, if an element is described in detail with reference to one embodiment and is not described with reference to a second embodiment, the element can nevertheless be claimed as included in the second embodiment.

As used herein, “proximal” and “distal” are spatial/directional terms that describe locations or directions based on their relationship to the two ends of a kinematic chain. “Proximal” is associated with the end of the kinematic chain that is closer to the base or support of the chain, while “distal” is associated with the opposite end of the kinematic chain, which often comprises an end effector of an instrument. When used in to refer to locations or to portions of a component, proximal and distal indicate the relative positions of the locations or portions relative to the base of the chain, with the proximal location or portion being closer to the base (closer here referring to proximity along the kinematic chain, rather than absolute distance). When used to refer to a direction, “proximal” refers to directions that point generally from a given location along a kinematic chain towards a more proximal location along the kinematic chain, and “distal” refers to directions that point from the given location towards a more distal location along the kinematic chain.

Unless otherwise noted herein or implied by the context, when terms of approximation such as “substantially,” “approximately,” “about,” “around,” “roughly,” and the like, are used in conjunction with a stated numerical value, property, or relationship, such as an end-point of a range or geometric properties/relationships (e.g., parallel, perpendicular, straight, etc.), this should be understood as meaning that mathematical exactitude is not required for the value, property, or relationship, and that instead a range of variation is being referred to that includes but is not strictly limited to the stated value, property, or relationship. In particular, the range of variation around the stated value, property, or relationship includes at least: any inconsequential variations; those variations that are typical in the relevant art for the type of item in question due to manufacturing or other tolerances; and/or variations that are within ±5% of the stated value, property, or relationship unless indicated otherwise.

As used herein, “transverse” refers to a positional relationship of two items in which one item is oriented crosswise at an angle relative to the other item, such as being substantially or generally perpendicular to the other item. As used herein, “transverse” includes, but does not require, an exactly perpendicular relationship.

Further modifications and alternative embodiments will be apparent to those of ordinary skill in the art in view of the disclosure herein. For example, the devices and methods can include additional components or steps that were omitted from the diagrams and description for clarity of operation. Accordingly, this description is to be construed as illustrative only and is for the purpose of teaching those skilled in the art the general manner of carrying out the present teachings. It is to be understood that the various embodiments shown and described herein are to be taken as exemplary. Elements and materials, and arrangements of those elements and materials, can be substituted for those illustrated and described herein, parts and processes can be reversed, and certain features of the present teachings can be utilized independently, all as would be apparent to one skilled in the art after having the benefit of the description herein. Changes can be made in the elements described herein without departing from the spirit and scope of the present teachings and following claims.

It is to be understood that the particular examples and embodiments set forth herein are non-limiting, and modifications to structure, dimensions, materials, and methodologies can be made without departing from the scope of the present teachings.

Other embodiments in accordance with the present disclosure will be apparent to those skilled in the art from consideration of the specification and practice of the invention disclosed herein. It is intended that the specification and examples be considered as exemplary only, with the following claims being entitled to their fullest breadth, including equivalents, under the applicable law.

1. A teleoperable manipulator system, comprising:
a table assembly comprising a platform configured to support a body, wherein the platform comprises lateral and longitudinal dimensions,
a rail coupled to the table assembly and comprising a floor-facing portion arranged to face a floor supporting the table assembly; and
a manipulator coupled to the rail and translatable relative to the rail along a longitudinal dimension of the rail,
wherein the manipulator comprises a proximal link assembly comprising a proximal arm coupled to the floor-facing portion of the rail by one or more proximal joints, and
wherein the one or more proximal joints are configured to swing the proximal arm with respect to the rail into and out of a space between the floor-facing portion of the rail and the floor.

2. The system of claim 1,
wherein the manipulator comprises an intermediate link assembly comprising an intermediate arm coupled to the proximal arm, and
wherein the intermediate arm is extendable in length.

3. The system of claim 1,
wherein the one or more proximal joints comprises:
a first proximal joint configured to provide rotation of the proximal arm about a first axis perpendicular to the longitudinal dimension of the rail.

4. The system of claim 3,
wherein the one or more proximal joints comprises a second proximal joint configured to provide rotation of the proximal arm about a second axis extending in a plane perpendicular to the first axis and parallel to the longitudinal dimension of the rail.

5. The system of claim 4,
wherein the proximal link assembly comprises a first joint housing and a second joint housing,
wherein the first joint housing is coupled to the bottom portion of the rail and rotatably coupled to the second joint housing by the first proximal joint,
wherein the second joint housing is rotatably coupled to the proximal arm by the second proximal joint, and
wherein the first and second joint housings comprise 90-degree bends.

6. (canceled)

7. The system of claim 1,
wherein the manipulator comprises
an intermediate link assembly comprising an intermediate arm coupled to the proximal arm,
a distal link assembly comprising a distal arm coupled to the intermediate arm,
a wrist, and
an instrument holding portion configured to mount an instrument thereto, and wherein the instrument holding portion is coupled to the distal arm by the wrist.

8. The system of claim 7,
wherein the wrist comprises three rotational degrees of freedom of motion.

9. The system of claim 8,
wherein the wrist comprises two wrist joints each configured to provide a differing one of the rotational degrees of freedom of motion, the wrist joints being driven by actuation elements coupled to actuators disposed remotely from the wrist.

10. The system of claim 9,
wherein the actuators are disposed in the distal arm.

11-88. (canceled)

89. The system of claim 1,
wherein the manipulator is configured to extend above and across the platform to a position adjacent a side of the platform opposite from a side on which the rail is disposed.

90. The system of claim 1, wherein the manipulator comprises:
an intermediate link assembly comprising an intermediate arm rotatably coupled to the proximal arm by a first intermediate joint,
wherein the manipulator is configured to position at least a portion of the first intermediate joint beneath the rail.

91. The system of claim 90,
wherein the manipulator is configured to position at least the proximal link assembly and the intermediate link assembly concurrently beneath the platform.

92. The system of claim 91, wherein the manipulator comprises:
a distal link assembly comprising a distal arm coupled to the intermediate arm; and
an instrument holding portion coupled to the distal arm;
wherein the manipulator is configured to enter a stowed configuration in which the proximal link assembly, the intermediate link assembly, the distal link assembly, and the instrument holding portion are concurrently positioned beneath the platform.

93. The system of claim 92,
wherein in the stowed configuration, the intermediate arm is laterally adjacent and extends parallel to the distal arm, and the distal arm is beneath the proximal arm.

94. The system of claim 1,
wherein the proximal arm has an asymmetrical shape such that, in extending between a proximal end portion of the proximal arm and a distal end portion of the proximal arm, a centerline of the proximal arm deviates from a straight line extending between the proximal end portion of the proximal arm and the distal end portion of the proximal arm.

95. The system of claim 94,
wherein the manipulator comprises an intermediate link assembly comprising an intermediate arm rotatably coupled to the proximal arm by an intermediate joint;
wherein the one or more proximal joints comprise a first proximal joint having a first axis of rotation and a second proximal joint having a second axis of rotation;
wherein the intermediate joint has a third axis of rotation parallel to the second axis of rotation; and
wherein the asymmetrical shape of the proximal arm is such that the first axis of rotation is offset from the second axis of rotation along a first direction and along a second direction orthogonal to the first direction, wherein the first direction is tangent to the centerline of the proximal arm at the second axis of rotation.

96. The system of claim 1,
where the rail is vertically fixed with respect to the platform.

97. The system of claim 1,
wherein the floor-facing portion is located on a plane parallel to the floor.

98. The system of claim 4,
wherein the proximal link assembly comprises a first link extending between the first and second proximal joints, and
wherein the proximal arm extends between the second proximal a third joint.

99. The system of claim 98,
wherein the third joint swings into and out of the space along an arc parallel to the floor.

100. A method of operating a medical system comprising a table assembly, a rail coupled to the table assembly, and a manipulator movably coupled to the rail, the method comprising:
moving a proximal arm of the manipulator about one or more proximal joints, which connect the proximal arm to a bottom portion of the rail facing a floor supporting the table assembly, such that at least a portion of the proximal arm is positioned beneath the rail.

101. The method of claim 100,
wherein the one or more proximal joints comprise a first proximal joint and a second proximal joint, and moving the proximal arm about the one or more proximal joints comprises:
rotating the proximal arm, via the first proximal joint, about a first axis perpendicular to a longitudinal dimension of the rail, and
rotating the proximal arm, via the second proximal joint, about a second axis extending in a plane perpendicular to the first axis and parallel to the longitudinal dimension of the rail.

102. The method of claim 100,
wherein the manipulator comprises an intermediate arm movably coupled to the proximal arm, a distal arm movably coupled to the intermediate arm, and an instrument holding portion movably coupled to the distal arm, and
wherein the method comprises moving the manipulator into a stowed configuration in which the proximal arm, the intermediate arm, the distal arm, and the instrument holding portion are concurrently positioned beneath a platform of the table assembly, the platform configured to support a body.
